(19) United States
(12) Patent Application Publication
(10) Pub. No.: US 2007/0150976 A1
(43) Pub. Date: Jun. 28, 2007

(54) High-level expression of fusion polypeptides in plant seeds utilizing seed-storage proteins as fusion carriers

(75) Inventors: Daichang Yang, Wuhan (CN); Kevin Hennegan, Denver, CO (US); Ning Huang, Davis, CA (US)

Correspondence Address: ARENT FOX PLLC, 1050 CONNECTICUT AVENUE, N.W., SUITE 400, WASHINGTON, DC, 20036, US

(73) Assignee: VENTRIA BIOSCIENCE, Sacramento, CA (US)

(21) Appl. No.: 10/585,976

(22) PCT Filed: Dec. 9, 2004
(86) PCT No.: PCT/US04/41083

§ 371 Date: Nov. 1, 2006

Related U.S. Application Data
(60) Provisional application No. 60/527,753, filed on Dec. 9, 2003.
(60) Provisional application No. 60/614,546, filed on Oct. 1, 2004.

(51) Int. Cl. A01H 5/00; C12N 15/82; C12N 5/04

(52) U.S. Cl. 800/278; 800/279; 800/280; 800/320; 800/320.1; 800/320.2; 800/320.3; 435/412; 435/468

(57) ABSTRACT

The expression of heterologous peptides or polypeptides in the seeds of monocot plants is optimized by generating fusion protein constructs in which monocot plant seed storage proteins are used as fusion protein carriers for the heterologous peptides or polypeptides. The heterologous peptides or polypeptides are preferably small, about 10 kDa or less and/or between 5 and 100 amino acids in length. These heterologous peptides or polypeptides may be used in human and animal nutritional and therapeutic compositions.

### CROSS-REFERENCE TO RELATED APPLICATIONS

This claims priority to U.S. Provisional Application No. 60/527,753, filed Dec. 9, 2003 and U.S. Provisional Application No. 60/614,546, filed Oct. 1, 2004. The contents of both applications are incorporated in their entirety herein by reference.

### FIELD OF THE INVENTION

The present invention relates to the expression of heterologous peptides or polypeptides in the seeds of monocot plants, such as rice plants, for use in making human and animal nutritional and therapeutic compositions. Expression is optimized by generating fusion protein constructs, wherein monocot plant seed storage proteins are utilized as fusion protein carriers for the heterologous peptides or polypeptides. The heterologous peptides or polypeptides are small, about 10 kDa or less, and are preferably between 5 and 100 amino acids in length.

### BACKGROUND OF THE INVENTION

Many heterologous peptides and polypeptides are in short supply due to the large quantities required for nutritional or therapeutic uses or due to the large demand of these heterologous peptides by the world population. Heterologous peptides and polypeptides that are less then 200 amino acids, but preferably between 5 and 100 amino acids in length, are useful for many applications, including antibody-binding epitopes, antimicrobial agents, AIDS, and cancer therapies and/or diagnostic assays for a variety of diseases. Further, certain heterologous peptides or polypeptides are required in large quantities to impart their biochemical and biological function. Expression of the heterologous peptides and polypeptides in monocot plants is a way of meeting the increased demand.

Chemical synthesis methods are typically used for production of heterologous peptide and polypeptide molecules. However, the specific amino acid sequence of some heterologous peptides may render it difficult or impossible to produce the heterologous peptide by chemical synthesis methods. For example, sequences containing consecutive isoleucine and valine residues, due to their bulky side chains, can form hydrophobic β-sheet structures that lead to aggregation of a given heterologous peptide chain when a target heterologous peptide is being chemically constructed on a resin-based matrix. This complexity of the chemical synthesis methods can substantially increase the cost structure of a given heterologous peptide and thereby create a commercial barrier.

An alternative is to develop a low-cost recombinant expression platform that represents a means of producing a heterologous peptide or polypeptide in commercial quantities. Creation of chimeric fusion proteins attaching the target heterologous peptide or polypeptide to a larger protein partner is one strategy for improving the production of these compounds in biological systems. The fusion partner increases the total size of the protein, thereby improving the expression levels on a mass basis and the potential stability of the target heterologous peptide or polypeptide.

Fusion strategies have been employed successfully in various systems, including bacterial, yeast and fungi, insect, and mammalian cells. Each host expression system has its associated advantages and disadvantages.

Historically, protein fusion systems in higher plants have been limited to using transit and/or signal peptides and N-terminal mature regions of endogenous plant proteins to effectively import foreign proteins into intracellular organelles or used for marker proteins such as GUS or GFP, which are utilized for monitoring, stabilizing and/or increasing selective plant gene expression.

As mentioned above, a substantial challenge facing the production of heterologous peptide or polypeptide products is the cost of production. Transgenic plants are attractive as hosts for the expression systems for compounds where large amounts of the product are needed to meet the expected demand. Advantages of transgenic crops include a lower capital investment, greater ease of scale up, and a low risk of pathogen contamination as transgenic plants are free from animal viruses and from toxins sometimes associated with microbial hosts. The level of expression of heterologous peptides and polypeptides has, however, been low and the purification process can be costly, making such an expression system commercially impracticable.

Thus, there is potential to increase the expression of heterologous peptide or polypeptide by utilizing the fusion approach. Prior to the present invention, monocot plant seed storage proteins have not been utilized as fusion carriers for heterologous peptides or polypeptides, although fusion proteins have been expressed in plants, for example, as disclosed in the references below, the contents of all of which are incorporated in their entirety by reference herein.

U.S. Pat. No. 5,292,646 discloses expression of soluble recombinant proteins by culturing a host cell to produce the fusion protein, which comprises a thioredoxin-like protein sequence fused to a selected heterologous peptide or protein, optionally containing a linker peptide providing a cleavage site.

U.S. Pat. No. 6,080,559 discloses expression of processed recombinant lactoferrin and lactoferrin polypeptide fragments from a fusion product in Aspergillus, by culturing a transformed Aspergillus fungal cell containing a recombinant plasmid.

WO 97/28272 discloses expression of authentic recombinant proteins from fusion proteins with additional domains and/or elements, such as Fc fragments, fused to the protein of interest by a polypeptide comprising a hinge region, a hydrophilic spacer, and a dibasic amino acid endoprotease cleavage site, wherein the spacer may be cleaved and then digested by carboxypeptidase B to yield the authentic protein.

U.S. Pat. No. 5,595,887 discloses the use of human carbonic anhydrase as a fusion carrier and affinity tag for small peptide molecules.

U.S. Pat. No. 5,686,079 discloses the expression in transgenic plants, particularly in transgenic tobacco plant leaves, of a fusion protein consisting of a small portion of the bacterial β-galactosidase (lac) protein and bacterial SpA protein. The expression level of the fusion protein was 0.002% by fresh weight of leaf tissue.

U.S. Pat. No. 5,767,372 discloses the expression in plants, particularly in transgenic tobacco callus and transgenic tobacco leaves, of a fusion protein consisting of an N-terminal portion of the bacterial npt II protein and the toxic portion of a Bt toxin polypeptide. The expression levels were extremely low for the fusion protein, at 25-50 ng/g (0.00005%) fresh weight of plant tissue.

U.S. Pat. No. 5,861,277 discloses the expression in transgenic Arabidopsis plants of a fusion protein consisting of an N-terminal portion of the Arabidopsis PAT1 protein and the bacterial GUS protein. The expression level of the fusion product was not detailed.

U.S. Pat. No. 5,929,304 discloses the expression in transgenic tobacco plants of human lysosomal enzymes incorporated into fusion protein constructs with a FLAG™ fusion peptide to facilitate purification. The expression of the fusion product for hGC (human glucocerebriosidase) was approximately 2.5 mg/1.6 Kg (0.0015%) fresh weight of tobacco leaf tissue.

U.S. Pat. No. 5,977,438 discloses the expression in infected tobacco plants of a fusion protein that includes a portion of the tobacco mosaic virus coat protein as fusion carrier coupled to a 12 amino acid peptide portion of a bacterial malarial surface antigen. This fusion protein was expressed in tobacco using a viral vector system and expression of the 12 amino acid peptide in tobacco leaves was obtained at 25 μg/gram (0.0004%) fresh weight of leaf tissue.

U.S. Pat. No. 6,018,102 discloses the prophetic construction of fusion proteins for expression in transgenic potato leaves and tubers where a plant ubiquitin protein portion is utilized as the carrier molecule for various small lytic peptides.

U.S. Pat. No. 6,288,304 discloses expression of somatotropin (growth hormone) in seeds of the oilseed crop Brassica napus, using a fusion protein consisting of the N-terminal region of the Brassica oil body protein oleosin as a fusion carrier.

U.S. Pat. No. 6,331,416 discloses prophetic constructs for expression of various fusion polypeptides in transgenic potato tubers. The N-terminal fusion carrier proposed is a bacterial cellulose binding domain (CBD) fused to any non-plant protein to obtain stable plant expression.

U.S. Pat. No. 6,448,070 discloses construction and expression of fusion proteins in plants, particularly isolated tobacco protoplasts or viral infected tobacco plants, where the fusion protein consists of an N-terminal portion of the alfalfa mosaic virus capsid protein and mammalian viral epitopes for HIV-1 and rabies. Levels of fusion protein expression were not detailed.

U.S. Pat. No. 6,455,759 discloses expression in transgenic angiosperm plants, e.g. tobacco, of a fusion strategy consisting of the two proteins, e.g. maker proteins luciferase and beta-glucuronidase (GUS), connected by a plant ubiquitin linking domain. Levels of expression of this fusion product have not been described.

U.S. Pat. Appl. Pub. No. 2002/0146779 discloses the use of fusion proteins for the high production of recombinant polypeptides with authentic amino-terminal amino acid in a variety of transgenic systems, including bacteria, yeasts, animals and plants. No data are given on the expression of any fusion proteins in plants or plant cells nor are any examples described of any chimeric gene fusion protein constructs expressed in plants.

U.S. Pat. Appl. Pub. No. 2003/0159182 discloses the use of signal-peptide fusion proteins for the production of herpes virus epitopes in the seeds of transgenic cereals, including rice. Plasmid constructs containing signal peptides for targeting of herpes surface antigens are detailed. An expression level of 0.5% total protein was obtained in rice seeds. No prophetic examples or data are given for utilizing monocot seed storage proteins as fusion carriers.

Schreier et al. (EMBO J 4, 25-32, 1985) disclose that transport of a bacterial neomycin phosphotransferase (npt) protein into tobacco chloroplasts in vitro is enhanced using a portion of the tobacco small subunit mature protein fused to npt.

Comai et a. (J. Biol. Chem. 263, 15104-15109, 1986) disclose that efficient transport of a bacterial 5-enolpyruvylshikimate-3-phosphate (ESP) synthase into tobacco chloroplasts in vitro and in vivo requires a fusion between the mature portion of the tobacco small subunit portion and a bacterial ESP synthase.

None of these patents or publications disclose high level expression of heterologous peptides or polypeptides in monocot plants using a monocot plant seed storage protein as a fusion carrier.

The use of transgenic plants as a production system is considered to be ideal for compounds where large amounts of the product are needed to meet expected demand. Advantages of transgenic crops include low capital investment, ease of scale-up, and low risk of pathogen contamination. A rice-based high-level expression system has been developed and successfully produced a variety of proteins.

One such protein is the trefoil factor family (TFF), which is comprised of three small peptides containing one or more ‘trefoil domains’. Each trefoil domain is comprised of approximately 40 amino acid residues. Each trefoil domain folds into three highly stable loops, each loop formed by one of the three cysteine-mediated disulfide bonds. These intrachain disulfide bonds form in a 1-5, 2-4 and 3-6 configuration depending on their order in the primary amino acid sequence.

All intestine trefoil factor (ITF) peptides are highly homologous. Human ITF consists of a 75 amino acid polypeptide. After cleavage of the N-terminal signal peptide, the resulting mature human ITF contains 60 amino acids. Human ITF is present in both monomer and dimer forms in gastrointestinal tissue.

The compact structure of the trefoil motif may be responsible for the marked resistance of trefoil peptides to proteolytic digestion, enabling them to remain viable in the harsh environment of the gastrointestinal tract. The single domain human ITF has seven cysteine residues, six of which are involved in maintaining the structure of the trefoil domain. The seventh cysteine residue is not part of the trefoil domain and is located three residues upstream of the C-terminus.

Several biological activities of ITF have been identified and include promotion of wound healing, stimulation of epithelial cell migration and protection of the small intestine epithelial barrier. Thus, ITF can be used in the prevention and treatment of a variety of disease conditions. A natural source of ITF is prepared from colonic and small intestinal mucosa, but the yield is very low and is unable to provide the large quantity of ITF necessary for clinical use in the prevention and treatment of the variety of disease conditions.

ITF has also been produced in yeast and recombinant plasmids, which were constructed to encode a fusion protein consisting of a hybrid leader sequence and mature ITF sequences. The leader sequence directs the fusion protein into the secretory (and processing) pathway of the yeast cell. As the expression level is about 100 mg/L, the overall quantity of ITF from these systems remains limited.

Another suitable protein is one that is involved with the human growth hormone (hGH). HGH has lipolytic/antilipogenic actions in vivo, which result in decreased fat mass, increased lean mass, and weight loss. In vitro and in vivo studies have indicated that this response is mediated in part by an increase in β-adrenorecptor coupling efficiency, increased activity of hormone-sensitive lipase, and an inhibitory effect on the action of insulin. The carboxy terminus of the hGH molecule (hGH 177-191{AOD9601}) has been identified as the lipid mobilizing domain of the intact hormone. This fragment inhibits the activity of acetyl-CoA carboxylase in adipocytes and hepatocytes, and it acts to reduce glucose incorporation into lipid in both isolated cells and tissues. A synthesized C-terminal fragment of hGH (AOD9604) contains a lipolytic domain that may be responsible for the lipolytic action of hGH. The parent molecule, AOD9601, induces lipolysis and fat oxidation in adipose tissue in vitro. In vivo, AOD9601 indices weight loss without affecting food intake as well as increasing lipolytic sensitivity and increasing fat oxidation with no adverse effects on insulin sensitivity.

The nature of the response to both hGH and AOD9604 is poorly understood. It is hypothesized that both molecules may influence the expression of the B3-andrenergic receptors (B3-ARs), the major lipolytic tissue in fat tissue. Both AOD9604 and hGH can increase B3-AR mRNA expression, as well as protein levels and function, in mouse and human cells lines in vitro. A mechanism for high level production of this peptide is critical for future use in any fat reduction therapy.

### SUMMARY OF THE INVENTION

One aspect of the invention includes a method for expression of heterologous peptide or polypeptide in monocot plant seeds, comprising fusing a heterologous peptide or polypeptide with a monocot seed storage protein in a monocot mature seed expression system, and expressing the heterologous peptide or polypeptide in the mature monocot seed.

Another aspect of the invention involves expression of the fusion construct to a level of at least 15-20 μg/grain in transgenic monocot seeds, a substantial (approximately 20-fold) improvement over expression of the heterologous peptide or polypeptide in the absence of any seed-storage protein fusion strategy. Expression of the fusion construct is preferably at least 3.0%, more preferably at least 5.0%, of total soluble protein in the grain.

Another aspect of the invention involves a highly successful fusion approach for the high-level expression of heterologous oligopeptide molecules by fusing a small polypeptide and a seed storage protein for expression in a mature monocot seed expression system.

Another aspect of the invention involves a strategic tryptophan residue providing a chemical cleavage site engineered ‘in frame’ between a seed storage protein and a small polypeptide. This site may be used for the release of the mature small polypeptide from the fusion carrier.

A further aspect of the invention includes a method for expression of a small (about 10 kDa or less and/or between 5 and 100 amino acids in length) heterologous peptide or polypeptide in monocot plant seeds, comprising fusing a small heterologous peptide or polypeptide with a monocot seed storage protein in a monocot mature seed expression system, and expressing the heterologous peptide or polypeptide in the mature monocot seed.

Another aspect of the invention is a fusion protein comprising an optional signal peptide, a monocot seed storage protein, and a small heterologous peptide or polypeptide. The monocot seed storage protein may be at the N-terminal or C-terminal side of the small heterologous peptide or polypeptide in the fusion protein. It is preferred that the monocot seed storage protein by located at the N-terminal side of the small heterologous peptide or polypeptide.

A further aspect of the invention is a fusion protein including a methionine or tryptophan residue engineered in frame between the small heterologous peptide or polypeptide and the monocot seed storage protein.

Another aspect of the invention comprises at least one selective purification tag and/or at least one specific protease cleavage site for eventual release of the heterologous peptide or polypeptide from the monocot seed storage protein carrier, fused in translation frame between the heterologous peptide or polypeptide and the monocot seed storage protein. Preferably, the specific protease cleavage site may comprise enterokinase (ek), Factor Xa, thrombin, V8 protease, Genenase™, α-lytic protease or tobacco etch virus (TEV) protease.

Another aspect of the present invention comprises cleavage of the fusion protein via chemical cleaving agents such as cyanogen bromide.

These and other aspects and features of the invention will become more fully apparent when the following detailed description of the invention is read in conjunction with the accompanying drawings.

### BRIEF DESCRIPTION OF THE FIGURES

FIG. 1 presents the comparison of the codon-optimized DNA sequence for the expression of the 60 amino acid mature portion of intestinal trefoil factor (ITF) in rice grains;

FIG. 2 presents the nucleotide and amino acid sequences for the constructed Gt1 signal peptide fused with the 19 kDa globulin protein (Glb) as a fusion carrier, the enterokinase (ek) cleavage site and the mature ITF protein all fused in the same translational reading frame;

FIG. 3 shows plasmid pAPI471 containing the chimeric-gene construct for the expression of the Glb-ek-ITF fusion protein in mature rice grains;

FIG. 4 shows the expression level of the Glb-ek-ITF fusion protein in mature rice grains;

FIG. 5 shows Western blot analysis of ITF expression as part of the Glb-ek-ITF fusion protein;

FIG. 6 indicates the comparison of the codon-optimized DNA sequence for the expression of the 16 amino acid AOD9604 (AOD) peptide in rice grains;

FIG. 7 indicates the nucleotide and amino acid sequences for the constructed Gt1 signal peptide fused with the 19 kDa globulin protein (Glb) as a fusion carrier, a cleavage site based on chemical cleavage of the amino acid tryptophan (designated #) and the AOD peptide all fused in the same translational reading frame;

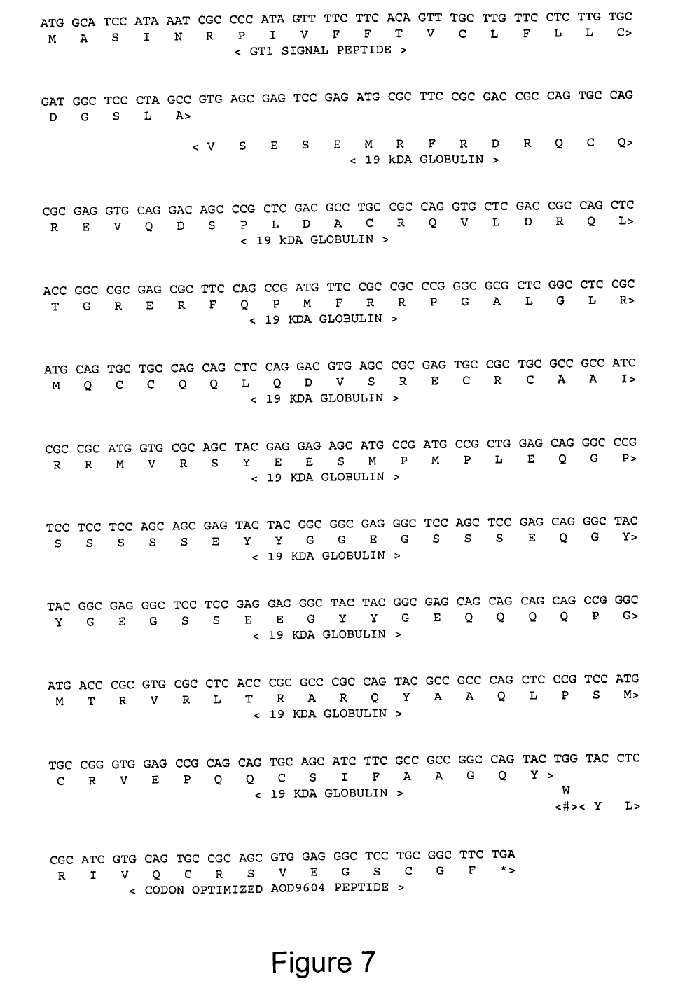

FIG. 8 shows plasmid pAPI507 containing the chimeric gene construct specifying the expression of the Glb-W-AOD fusion protein in mature rice grains;

FIG. 9 shows the DNA and amino acid sequences of the N-terminal region of the globulin-M-AOD9604 fusion polypeptide;

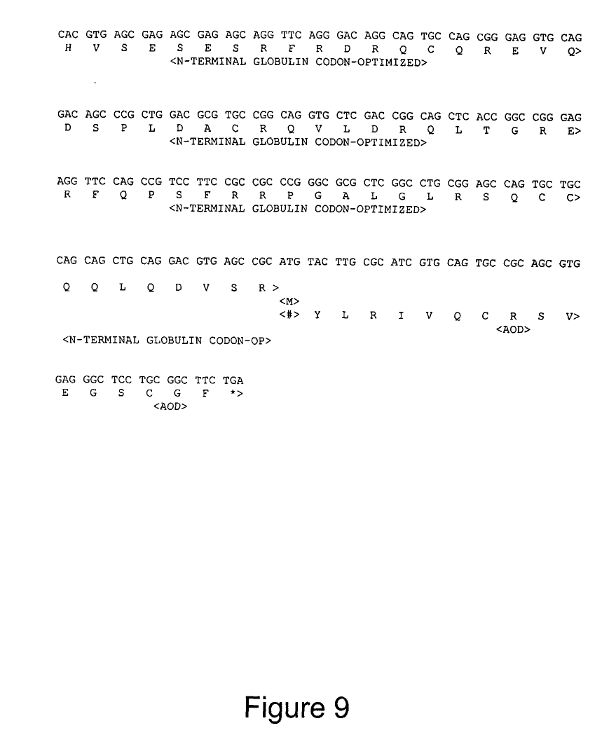

FIG. 10 shows the DNA and amino acid sequences of the His6-mutated globulin-M-AOD9604 polypeptide;

FIG. 11 shows plasmid pPAI502;

FIG. 12 shows plasmid pAPI499;

FIG. 13 shows AOD9604 fusion identity confirmation by Western blot analysis using fusion partner—and AOD9604-specific antibody. Total protein was extracted with 66 mM Tris-HCl, pH 6.8, 2% SDS and 2% β-mercaptoethenal. Panel A indicates the SDS-PAGE coomassie staining gel. Panel B and C present the results of western blot analysis using antiserum against AOD9604 and globulin, respectively. Lane 1 shows the negative control, TP309, lane 2 and 3 indicate the transgenic line 507-13. Lane 4 shows the transgenic line 507-17. Twenty milliliters of total protein extraction buffer was used to extract one gram transgenic flour and 15 μl of extract was loaded;

FIG. 14 shows the SDS-PAGE Coomassie-staining gel of the top seven lines expressing AOD9604 fusion protein from the pAPI499 construct. One gram of rice flour from the first generation of brown seeds was extracted with 25 ml of TBS plus 0.5M NaCl for 2 h. The slurry was centrifuged for 20 min at 5,000 rpm. The supernatant was discarded and the pellet was extracted with 15 ml of 2% SDS and 0.2% beta-mercaptoethanol. One milliliter of extract was removed and centrifuged at 14,000 rpm for 12 min. 35 μl of supernatant was loaded and separated on 4-20% SDS-PAGE gel. The gel was stained with Coommassie blue staining solution;

FIG. 15 shows Western blotting of the nGLB-AOD fusion protein. One gram of rice flour from first generation seeds was extracted with 25 ml of TBS plus 0.5M NaCl for 2 h. The slurry was centrifuged for 20 min at 5000 rpm. The supernatant was discarded and the pellet was extracted with 15 ml of 2% SDS and 2% beta-mercaptoethanol. One milliliter of extract was removed and centrifuged at 14000 rpm for 12 min. 40 μl of supernatant was loaded;

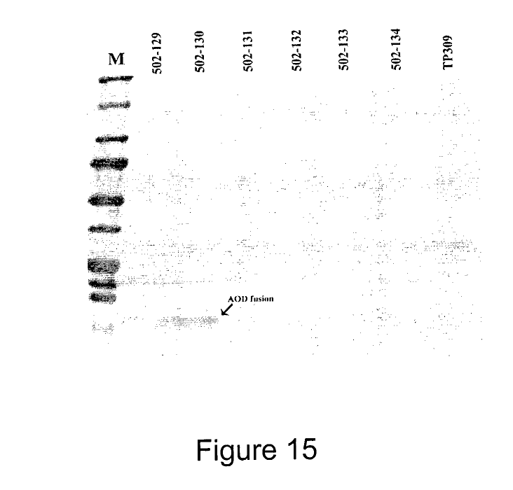

FIG. 16 shows the comparison of codon-optimization of insulin-like growth factor (IGF-1 opt) to native IGF-1.

FIG. 17 shows the DNA and amino acid sequences of GLB-W-IGF.

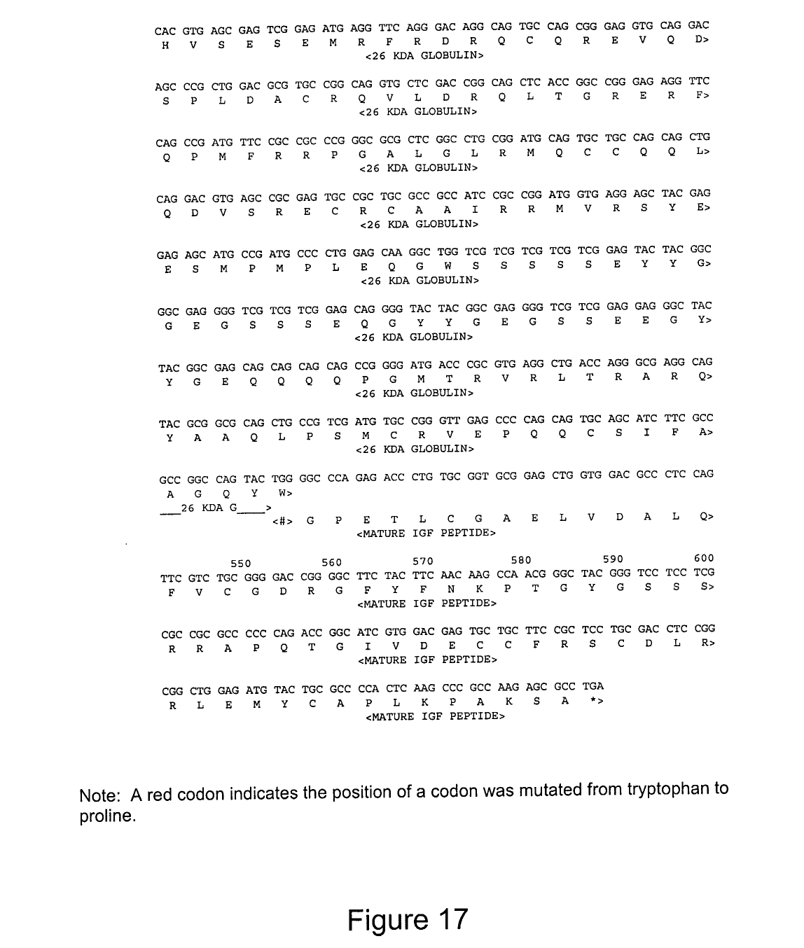

FIG. 18 the DNA and amino acid sequences of the basic subunit of glutelin-W-IGF.

FIG. 19 shows plasmid pAPI520; and

FIG. 20 shows plasmid pAPI521.

### DETAILED DESCRIPTION

Unless otherwise indicated, all terms used herein have the meanings given below or are generally consistent with the same meaning that the terms have to those skilled in the art of the present invention.

As used herein, the term “seed” refers to all seed components, including, for example, the coleoptile and leaves, radicle and coleorhiza, scutulum, starchy endosperm, aleurone layer, pericarp and/or testa, either during seed maturation and seed germination. In the context of the present invention, the term “seed” and “grain” is used interchangeably.

The term “biological activity” refers to any biological activity typically attributed to that protein by those of skill in the art.

The terms “fusion carrier” and “fusion partner” are used interchangeably, as understood by those of ordinary skill in the art.

The “heterologous peptide or polypeptide” comprises a coding sequence for a heterologous peptide or polypeptide of interest. The heterologous peptide or polypeptide of interest is preferably less then 200 amino acids in length. Preferably a small heterologous peptide or polypeptide is used in accordance with the invention, which is about 10 kDa or less and/or comprises 5 to 100 amino acids. For example, the 60 amino acid intestinal trefoil factor may be utilized as a small heterologous peptide or polypeptide.

Other heterologous peptides and polypeptides of interest are of mammalian origin. Such heterologous peptides and polypeptides include, but are not limited to, milk proteins, blood proteins (such as, serum albumin, Factor VII, Factor VIII or modified Factor VIII, Factor IX, Factor X, tissue plasminogen factor, Protein C, von Willebrand factor, antithrombin III, and erythropoietin), colony stimulating factors (such as, granulocyte colony-stimulating factor (G-CSF), macrophage colony-stimulating factor (M-CSF), and granulocyte macrophage colony-stimulating factor (GM-CSF)), cytokines (such as, interleukins), integrins, addressins, selectins, homing receptors, surface membrane proteins (such as, surface membrane protein receptors), T cell receptor units, immunoglobulins, soluble major histocompatibility complex antigens, structural proteins (such as, collagen, fibroin, elastin, tubulin, actin, and myosin), growth factor receptors, mammalian growth factors, growth hormones, cell cycle proteins, vaccines, fibrinogen, thrombin, cytokines, hyaluronic acid and antibodies.

The term “mammalian growth factor” refers to proteins, or biologically active fragments thereof, including, without limitation, epidermal growth factor (EGF), keratinocyte growth factors (KGF) including KGF-1 and KGF-2, insulin-like growth factors (IGF) including IGF-I and IGF-II, intestinal trefoil factor (ITF), transforming growth factors (TGF) including TGF-β and -β-3, granulocyte colony-stimulating factor (GCSF), nerve growth factor (NGF) including NGF-β, and fibroblast growth factor (FGF) including FGF-1-19 and -12 β, and biologically active fragments of these proteins. The sequences of these and other human growth factors are well-known to those of ordinary skill in the art. In a preferred embodiment of the present invention, the mammalian growth factor is ITF. It is even more preferred that the expression level in monocot plant seeds of ITF is 15-20 μg/grain.

The term “milk protein” refers to proteins, or biologically active fragments thereof, including, without limitation, lactoferrin, lysozyme, lactoferricin, epidermal growth factor, insulin-like growth factor-1, lactohedrin, kappa-casein, haptocorrin, lactoperoxidase, immunoglogulins, and alpha-1-antitrypsin. Preferably, the milk proteins are lysozyme or lactoferrin.

While a peptidic product will generally be the result, genes may be introduced which may serve to modify non-peptidic products produced by the cells. These heterologous peptides or polypeptides, and fragments thereof, usually of at least 10 amino acids, fused combinations, mutants, and synthetic peptides or polypeptides, whether the peptides or polypeptides may be synthetic in whole or in part, so far as their sequence in relation to a natural peptide or polypeptide, may be produced as well.

In addition, this successful method to attain high-level expression of heterologous peptide or polypeptide in monocot seeds allows for the expression of a variety of other heterologous peptides or polypeptides of nutritional or therapeutic importance. These include, but are not limited to: peptides for treating obesity such as AOD9604 and PYY, potential peptide antibiotics such as iseganan and β-defensin, mature peptide growth factors such as EGF, IGF and FGF, anti-HIV peptides such as Fuzeon and its derivatives, peptide hormones and peptide hormone fragments such as parathyroid hormone (PTH), adrenocorticotropin (ACTH) and gastrin-releasing peptide (GRP) and peptides for treating hypertension such as vasoactive intestinal peptide (VIP) and vascular endothelial growth inhibitor (VEGI).

Further, heterologous peptides and polypeptides for human or veterinary use, such as vaccines and growth hormones, may be produced. The monocot plant seeds containing the polypeptide of interest can be formulated into mash product or formulated seed product directly useful in human or veterinary applications.

Due to the inherent degeneracy of the genetic code, however, a number of nucleic acid sequences which encode substantially the same or a functionally equivalent amino acid sequence may be generated and used to clone and express a given heterologous peptide or polypeptide. Thus, for a given heterologous peptide or polypeptide encoding nucleic acid sequence, it is appreciated that as a result of the degeneracy of the genetic code, a number of coding sequences can be produced that encode the same protein amino acid sequence. Such substitutions in the coding region fall within the range of sequence variants covered by the present invention. Any and all of these sequence variants can be utilized in the same way as described herein for the exemplified heterologous peptide or polypeptide encoding nucleic acid sequence.

As will be understood by those of skill in the art, in some cases it may be advantageous to use a heterologous peptide or polypeptide encoding nucleotide sequences possessing non-naturally occurring codons. Codons preferred by a particular eukaryotic host can be selected, for example, to increase the rate of expression or to produce recombinant RNA transcripts having desirable properties, such as a longer half-life, than transcripts produced from naturally occurring sequence. As an example, it has been shown that codons for genes expressed in rice are rich in guanine (G) or cytosine (C) in the third codon position. Changing low G+C content to a high G+C content has been found to increase the expression levels of foreign protein genes in barley grains. The DNA sequences employed in the present invention may be based on the rice gene codon bias along with the appropriate restriction sites for gene cloning.

“Seed maturation” refers to the period starting with fertilization in which metabolizable reserves, e.g., sugars, oligosaccharides, starch, phenolics, amino acids, and proteins, are deposited, with and without vacuole targeting, to various tissues in the seed (grain), e.g., endosperm, testa, aleurone layer, and scutellar epithelium, leading to grain enlargement, grain filling, and ending with grain desiccation.

The promoters useful in the present invention are any promoters that are active in plant cells. The type of promoter used is not critical, and does not make up the novel features of the invention. A preferred type of promoter is a promoter from the gene of a maturation-specific monocot seed storage protein (a.k.a. “maturation-specific protein promoter”). “Maturation-specific protein promoter” refers to a promoter exhibiting substantially upregulated activity (greater than 25%) during seed maturation.

A “signal sequence” or a “signal peptide” (used interchangeably) is an N- or C-terminal polypeptide sequence, which is effective to localize the peptide or protein to which it is attached to a selected intracellular or extracellular region, such as seed endosperm, or to transport the peptide or protein from the cell. The type of signal sequence used is not critical, and does not make up the novel features of the invention. Preferably, the signal sequence targets the attached peptide or protein to a location such as an endosperm cell, more preferably an endosperm-cell subcellular compartment or tissue, such as an intracellular vacuole or other protein storage body, chloroplast, mitochondria, or endoplasmic reticulum, or extracellular space, following secretion from the host cell.

As used herein, the terms “native” or “wild-type” relative to a given cell, polypeptide, nucleic acid, trait or phenotype, refers to the form in which that is typically found in nature.

As used herein, the term “purifying” is used interchangeably with the term “isolating” and generally refers to any separation of a particular component from other components of the environment in which it is found or produced. For example, purifying a recombinant protein from plant cells in which it was produced typically means subjecting transgenic protein-containing plant material to separation techniques such as sedimentation, centrifugation, filtration, column chromatography. The results of any of such purifying or isolating steps may still contain other components as long as the results have less other components (“contaminating components”) than before such purifying or isolating steps.

As used herein, the terms “transformed” or “transgenic” with reference to a host cell means the host cell contains a non-native or heterologous or introduced nucleic acid sequence that is absent from the native host cell.

The term “operably linked” as used herein, means that a nucleic acid is placed into a functional relationship with another nucleic acid sequence. For example, a promoter is operably linked to a coding sequence if it affects the transcription of the sequence. Linking is accomplished by ligation at convenient restriction sites. If such sites do not exist, synthetic oligonucleotide adaptors or linkers are used in accordance with conventional practice.

The terms “monocot seed storage protein” or “maturation specific monocot seed storage protein” (used interchangeably) refer to proteins, or biologically active fragments thereof, including, without limitation, globulin, rice glutelins, oryzins, prolamines, barley hordeins, wheat gliadins and glutenins, maize zeins and glutelins, oat glutelins, sorghum kafirins, millet pennisetins, or rye secalins.

In a preferred embodiment of the present invention, the monocot seed storage protein is 19 kilodalton (kDa) globulin from rice. The globulin gene has been isolated, characterized and the DNA sequence determined. Two dimensional gel electrophoresis of rice seed storage protein extracts indicates that the 19 kilodalton (kDa) globulin protein is largely, if not entirely, a single component and does not appear to exist as a family of proteins. Although the content in rice endosperm of the 19 kDa globulin protein is roughly 10% of the glutelin protein content, the 19 kDa globulin protein may be the most abundant product of a single gene in rice endosperm and in this respect, it is an excellent choice to manipulate as a fusion carrier for heterologous peptide expression in the rice endosperm.

In a further embodiment, the present invention allows for high-level expression of a heterologous antigenic polypeptide epitope specific for a variety of bacterial and viral diseases that could be used for oral immunization of these diseases.

Thus, the present invention provides a highly successful fusion approach for optimizing expression of heterologous peptides or polypeptides by fusing a heterologous peptide or polypeptide with a monocot seed storage protein in a monocot mature seed expression system. In one preferred embodiment, the present invention provides for fusion of a small polypeptide, e.g. intestinal trefoil factor (ITF), with a rice seed storage protein, e.g. globulin (Glb), in a rice mature seed expression system.

Optionally, at least one selective purification tag and/or specific peptide cleavage site can be engineered in the translation frame between the monocot seed storage protein and the heterologous peptide or polypeptide. In a preferred embodiment, a synthetic oligonucleotide encoding a peptide cleavage site for human enterokinase (ek) is engineered ‘in frame’ between the globulin and ITF protein domains. This site can be utilized for potential release of the mature ITF protein from the globulin fusion carrier.

Expression vectors for use in the present invention are chimeric nucleic acid constructs (or expression vectors or cassettes), designed for operation in plants, including appropriate associated upstream and downstream sequences.

In general, expression vectors for use in practicing the invention may include the following operably linked components that constitute a chimeric gene: (a) a promoter from the gene of a maturation-specific monocot seed storage protein; (b) an optional first DNA sequence, operably linked to said promoter, encoding a monocot plant seed-specific signal sequence capable of targeting a heterologous peptide or polypeptide linked thereto to a monocot plant seed storage body; (c) a second DNA sequence, encoding a monocot seed storage protein; and (d) a third DNA sequence, encoding a heterologous peptide or polypeptide, wherein the first, second, and third DNA sequences are linked in translation frame and together encode a fusion protein comprising the optional signal sequence, the storage protein, and the heterologous peptide or polypeptide.

The chimeric gene, in turn, may be placed in a suitable plant-transformation (“expression”) vector having (i) companion sequences upstream and/or downstream of the chimeric gene which are of plasmid or viral origin and provide necessary characteristics to the vector to permit the vector to move DNA from one host to another, such as from bacteria to a desired plant host; (ii) a selectable marker sequence; and (iii) a transcriptional termination region with or without a polyA tail.

Exemplary methods for constructing chimeric genes and transformation vectors carrying the chimeric genes are given in the examples below.

In the present invention, a heterologous polynucleotide can be expressed under the control of a promoter from a transcription initiation region that is preferentially expressed in plant seed tissue. Exemplary preferred promoters include a glutelin (Gt1) promoter, which effects gene expression in the outer layer of the endosperm and a globulin (Glb) promoter, which effects gene expression in the center of the endosperm. Promoter sequences for regulating transcription of gene coding sequences operably linked thereto include naturally-occurring promoters, or regions thereof capable of directing seed-specific transcription, and hybrid promoters, which combine elements of more than one promoter. Methods for construction such hybrid promoters are well known in the art.

In some cases, the promoter is derived from the same plant species as the plant cells into which the chimeric nucleic acid construct is to be introduced. Promoters for use in the invention are typically derived from cereals such as rice, barley, wheat, oat, rye, corn, millet, triticale or sorghum. Alternatively, a seed-specific promoter from one type of plant may be used to regulate transcription of a nucleic acid coding sequence from a different plant.

Further examples of promoters useful to the present invention include, but are not limited to, a maturation-specific promoter associated with one of the following maturation-specific monocot storage proteins listed above. Also included are aleurone and embryo specific promoters associated with the rice, wheat and barley genes such as lipid transfer protein Ltp1, chitinase Chi26, and Em protein Emp1.

Other promoters suitable for expression in maturing seeds include the barley endosperm-specific B1-hordein promoter, GluB-2 promoter, Bx7 promoter, Gt3 promoter, GluB-1 promoter and Rp-6 promoter. Preferably, these promoters are used in conjunction with transcription factors.

In addition to encoding the protein of interest, the expression cassette or heterologous nucleic acid construct may encode a signal peptide that allows processing and translocation of the protein, as appropriate. Exemplary signal sequences, defined supra, are signal sequences associated with the monocot maturation-specific genes: glutelins, prolamines, hordeins, gliadins, glutenins, zeins, albumin, globulin, ADP glucose pyrophosphorylase, starch synthase, branching enzyme, Em, and lea.

Further, as many monocot seed storage proteins are under the control of a maturation-specific promoter and this promoter is operably linked to a leader sequence for targeting to a protein body, the promoter and leader sequence can be isolated from a single protein-storage gene, operably linked to a heterologous peptide or polypeptide in a chimeric gene construct. One exemplary promoter-leader sequence is from the rice Gt1 gene. Alternatively, the promoter and leader sequence may be derived from different genes, e.g. the rice Glb promoter linked to the rice Gt1 leader sequence.

Production of the heterologous peptide or polypeptide can be enhanced by codon optimization of the gene. The intent of codon optimization was to change an A or T at the third position of the codons of G or C. This arrangement conforms more closely with codon usage in typical rice genes. Such codon optimization is intended to be within the scope of the present invention.

Suitable selectable markers for selection in monocot plant cells include, but are not limited to, antibiotic resistance genes, such as kanamycin (nptII), G418, bleomycin, hygromycin, chloramphenicol, ampicillin, tetracycline, and the like. Additional selectable markers include a bar gene which codes for bialaphos resistance; a mutant EPSP synthase gene which encodes glyphosate resistance; a nitrilase gene which confers resistance to bromoxynil; a mutant acetolactate synthase gene (ALS) which confers imidazolinone or sulphonylurea resistance. The particular marker gene employed is one which allows for selection of transformed cells as compared to cells lacking the DNA which has been introduced. Preferably, the selectable marker gene is one that facilitates selection at the tissue culture stage, e.g., a nptII, hygromycin or ampicillin resistance gene. Thus, the particular marker employed is not essential to this invention.

In general, a selected nucleic acid sequence is inserted into an appropriate restriction endonuclease site or sites in the vector. Standard methods for cutting, ligating and E. coli transformation, known to those of skill in the art, are used in constructing vectors for use in the present invention.

Plant cells or tissues are transformed with above expression constructs using a variety of standard techniques. It is preferred that the vector sequences be stably integrated into the host genome.

To be “stably transformed” in the context of the present invention means that the introduced nucleic acid sequence is maintained through two or more generations of the host, which is preferably (but not necessarily) due to integration of the introduced sequence into the host genome. The method used for transformation of host plant cells is not critical to the present invention. For commercialization of heterologous peptide or polypeptide expressed in accordance with the present invention, the transformation of the plant is preferably permanent, i.e. by integration of the introduced expression constructs into the host plant genome, so that the introduced constructs are passed onto successive plant generations. The skilled artisan will recognize that a wide variety of transformation techniques exist in the art, and new techniques are continually becoming available.

Any technique that is suitable for the target host plant may be employed within the scope of the present invention. For example, the constructs can be introduced in a variety of forms including, but not limited to, as a strand of DNA, in a plasmid, or in an artificial chromosome. The introduction of the constructs into the target plant cells can be accomplished by a variety of techniques, including, but not limited to calcium-phosphate-DNA co-precipitation, electroporation, microinjection, Agrobacterium-mediated transformation, liposome-mediated transformation, protoplast fusion or microprojectile bombardment. The skilled artisan can refer to the literature for details and select suitable techniques for use in the methods of the present invention.

Transformed plant cells are screened for the ability to be cultured in selective media having a threshold concentration of a selective agent. Plant cells that grow on or in the selective media are typically transferred to a fresh supply of the same media and cultured again. The explants are then cultured under regeneration conditions to produce regenerated plant shoots. After shoots form, the shoots can be transferred to a selective rooting medium to provide a complete plantlet. The plantlet may then be grown to provide seed, cuttings, or the like for propagating the transformed plants. The method provides for efficient transformation of plant cells with expression of a gene of heterologous origin and regeneration of transgenic plants, which can produce a heterologous peptide or polypeptide.

The expression of the heterologous peptide or polypeptide may be confirmed using standard analytical techniques such as Western blot, ELISA, PCR, HPLC, NMR, or mass spectroscopy, together with assays for a biological activity specific to the particular protein being expressed.

The expression systems described in the Examples below are based on specific sequence systems. However, one of skill in the art will appreciate that the invention is not limited to a particular system. Thus, in other embodiments, other promoters and other signal sequences may be employed to express heterologous peptides or polypeptides in monocot plant seeds.

### EXAMPLE 1

### Human ITF Sequence and Plasmid Construction

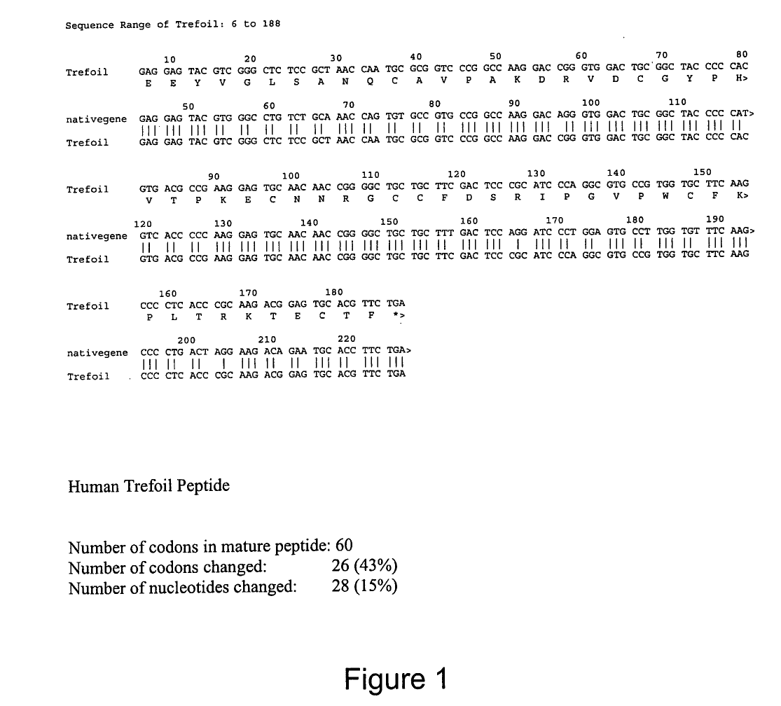

Human ITF DNA sequence was based on the GenBank accession number L08044. This sequence encodes an open reading frame of 75 amino acid ITF peptide. For expression of mature ITF in rice grains, the DNA sequence encoding the 60 amino acid mature ITF peptide was codon-optimized (ITF, FIG. 1) based on a codon-table specific for the expression of endogenous rice genes.

FIG. 1 shows the comparison of the codon-optimized DNA sequence for the expression of the 60 amino acid mature portion of intestinal trefoil factor (ITF) in rice grains. ‘Native genes’ refers to the normal human ITF DNA sequence while ‘Trefoil’ refers to the codon-optimized ITF DNA sequence. The corresponding amino acid sequence is listed below the DNA sequence.

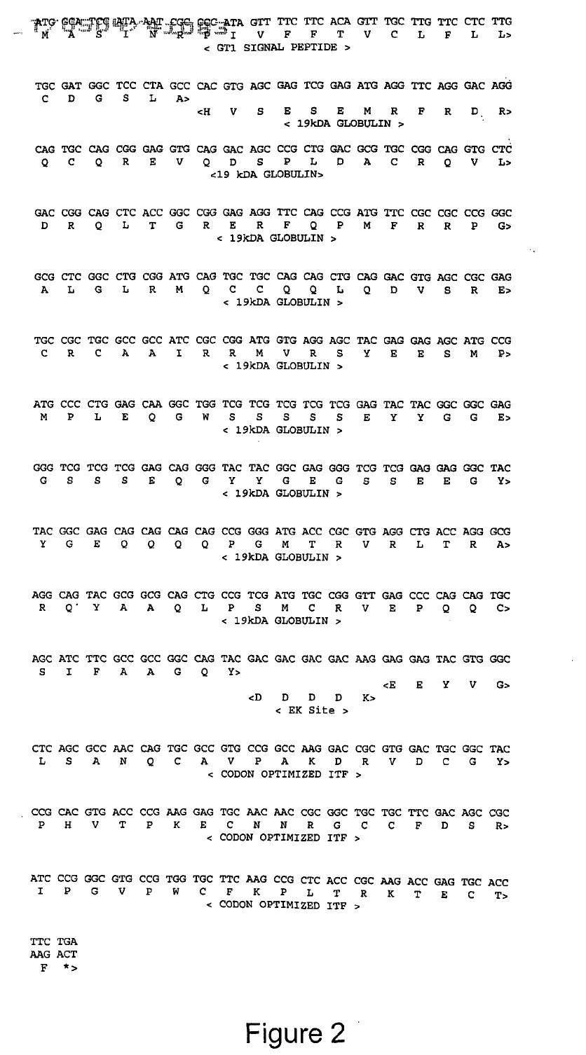

FIG. 2 presents the nucleotide and amino acid sequences for the constructed Gt1 signal peptide fused with the 19 kDa globulin protein (Glb) as a fusion carrier, the enterokinase (ek) cleavage site and the mature ITF protein all fused in the same translational reading frame.

The codon-optimized ITF gene encoding mature ITF was derived by chemical synthesis and cloned into the Stratagene universal cloning vector pCR2.1 via single strand DNA amplification and the A/T overhang method. This resulting plasmid was designated pAPI431.

Plasmid pAPI471 was ultimately constructed utilizing three intermediate plasmids: a rice globulin fusion partner (pAPI469), the ek (enterokinase) linker-ITF (pAPI465) and the rice codon-optimized ITF gene described above (pAPI431). The fusion partner, the 19 kDa rice globulin gene, was amplified via primer pairs designed from GenBank accession No.X63990 and cloned into the Stratagene pCR2.1 vector. The amplified and cloned DNA sequences encoding the 19 kD globulin were confirmed by DNA sequencing analysis. This resulting plasmid was called pAPI469. Next, a 15 base pair enterokinase (ek) linker DNA segment was introduced into pAPI431 via site-directed mutagenesis on the N-terminal coding region of the mature codon-optimized ITF. The resulting plasmid, pAPI465 contains ek-ITF gene fusion.

Plasmid pAPI469 was digested with the enzymes HindIII and SnaBI and then cloned into pAPI465 which was digested by Mfel (blunted by Mung bean nuclease) and HindIII. The two DNA segments were isolated on a 1% agarose gel and purified using QIAGEN gel extraction protocol. The two fragments were ligated with T4 DNA ligase and used to transform competent E. coli cells. The resulting plasmid contained the gene encoding the 19 kD globulin-ek-codon-optimized ITF fusion. This intermediate plasmid was designated pAPI470.

The DNA fragment containing the Glb-ek-ITF obtained from pAPI470 was digested by BamHI (blunted by Mung bean nuclease) and Xhol and cloned into the NaeI and XhoI sites of pAPI405. Both DNA segments of pAPI405 and pAPI470 digests were purified from 1% agarose gels and ligated. Plasmid pAPI405 is a derivative of the rice Gt1 promoter cassette vector pAPI141 and contains the Gt1 promoter, the Gt1 signal peptide and the nos terminator region. The linker region between the Gt1 promoter and nos terminator in pAPI405 contains a 1.8 Kb Gus gene stuffer fragment. The resulting pAPI471 plasmid contains the rice Gt1 promoter, the rice Gt1 signal peptide, the rice globulin protein as the fusion carrier, the enterokinase cleavage site fused in frame to the codon-optimized ITF gene (Gt1 promoter/Gt1sg-Glb-ek-ITF), and the nos terminator region.

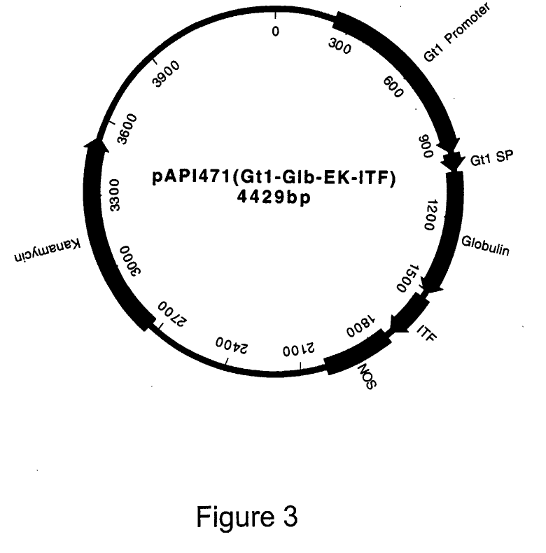

FIG. 3 shows plasmid pAPI471 containing the chimeric-gene construct for the expression of the Glb-ek-ITF fusion protein in mature rice grains. Expression of the fusion protein is under the control of the rice Gt1 promoter as indicated. Kanamycin refers to the bacterial selectable marker on the plasmid. Relevant restriction enzyme sites are noted.

### EXAMPLE 2

### Rice Transformation and Plant Regeneration

A selectable marker plasmid pAPI176, consisting of the hygromycin B phosphotransferase (Hph) gene driven by the Gns9 promoter and followed by a NOS terminator, provided the selectable marker DNA segment for all plant transformations. Plasmid DNA was digested with appropriate enzymes to linearize the DNA and was then separated by 1% low melting agarose gel. After separation, the DNA fragment was eluted from the agarose gel slices and the agarose was removed by digestion with Agarase.

The DNA was precipitated and run on a gel to check for linear DNA purity with respect to intact plasmid DNA. A total of 50 μl of gold particles were coated with 0.65 μg DNA and the DNA amounts of the selected marker fragment and target gene fragment were calculated at a molar ratio of 1:1. Rice calli obtained from immature rice embryos were prepared for transformation as described by Huang et al. (Molec. Breeding 10, 83-94, 2001). Microprojectile-projectile mediated transformation of rice was carried out according to the procedure described by Huang et al. Transgenic rice plants were raised to maturity in the greenhouse and their seeds were harvested.

### EXAMPLE 3

### Analysis of ITF-Containing Fusion Protein Expression in Mature Rice Grains

For protein extraction, individual dehusked rice grains from transgenic plants containing the construct of ITF-fusion protein were placed in the wells of a grinding plate. Each well was given 0.2 ml of extraction buffer, Tris-buffered saline (TBS) plus 0.35M NaCl. The grains were ground using a Genome Grinder for 12 minutes at 1300 strokes per minute. The resulting seed extracts were centrifuged at 4000 rpm for 20 minutes and the seed supernatants were transferred to a new plate.

Alternatively, 10 dehusked rice grains were pooled and ground with a mortar and pestle in 2 ml of extraction buffer, TBS plus 0.35M NaCl, and then mixed for 1.5 hours at 37° C. The mixed slurry was centrifuged at 12000 rpm for 12 minutes and the supernatant was transferred to a 2 ml Eppendrof tube and stored at −20° C. for future analysis.

For expression level analysis, a total of 32 μl (approximately 50-60 μg total protein) of individual seed supernatants were resolved on 4-20% precast polyacrylamide gels (Novex, Carlsbad, Calif.). The gel was stained with staining solution, 0.1% Coomassie Brilliant Blue R-250, and then destained to visualize protein bands. For Western blot analysis, the gel was electroblotted to a 0.45 μm nitrocellulose membrane, blocked with 5% non-fat dry milk in phosphate-buffered saline (PBS) for three hours and then rinsed in PBS. For incubation with primary antibody, a mouse monoclonal antibody against ITF (GI Laboratories) was used at 1:1000 dilution in a primary antibody solution, 5% BSA in PBS containing 0.05% Tween20. The blot was incubated in the solution overnight.

The resulting blot was washed with PBS three times for 10 minutes each time. The secondary antibody (goat anti-rabbit IgG-alkaline phosphatase conjugate (Bio-Rad, CA)) was 1:4000 diluted in blocking buffer. The membrane was then incubated in the secondary antibody solution for two hours and then washed three times in PBS. Color development was initiated by adding the substrate BCIP-NBT (Sigma, St. Louis, Mo.), and the process was terminated by rinsing the blot with water once the desired intensity of the bands was achieved.

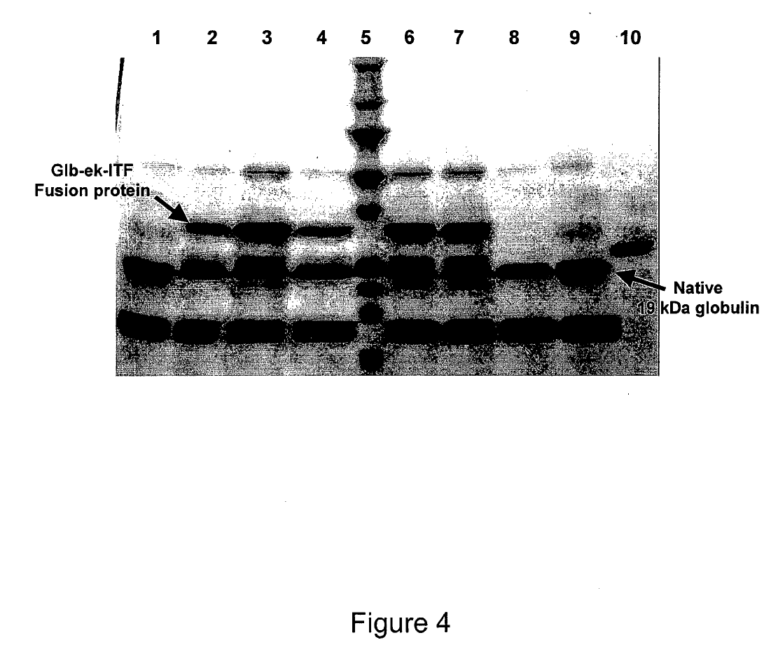

FIG. 4 shows the expression of Glb-ek-ITF fusion protein resolved by Coomassie stained PAGE. Approximately 50-60 μg of individual R1 generation seed protein extracts were prepared from transgenic rice event 471-70 and resolved on 4-20% PAGE. Lane 1 refers to control extract from the non-transgenic rice variety Tapei 309 (TP309). Extracts from seven segregating individual seeds of the 471-70 transformation event are shown—lanes 2-4 and 6-9. Molecular weight markers are displayed in lane 5. For estimating the amount of fusion protein present, approximately 5 μg of a marker protein, the 23 kDa carbonic anhydrase (Sigma) was loaded in the gel (lane 10) as an expression level reference. It is estimated that lanes 471-70-2, 471-70-4 and 471-70-5 contain Glb-ek-ITF fusion protein bands of approximately 10 μg. The positions of the endogenous or native 19 kDa globulin protein and the approximately 28 kDa Glb-ek-ITF fusion protein are indicated by arrows. This band corresponding to Glb-ek-ITF fusion protein, indicated by the arrow, is not present in control TP309.

Since one-sixth of the volume of the seed extract volume was loaded onto the gel, the total fusion protein is estimated to be about 60 μg/grain or 0.3% of total grain weight. About 300 to 400 μg of total protein per grain is generally extracted with the extract buffer, so the recombinant fusion protein is about 15 to 20% of total soluble protein. ITF is about one fourth of the fusion protein by weight, so ITF is about 15 μg/grain or 0.075% grain weight.

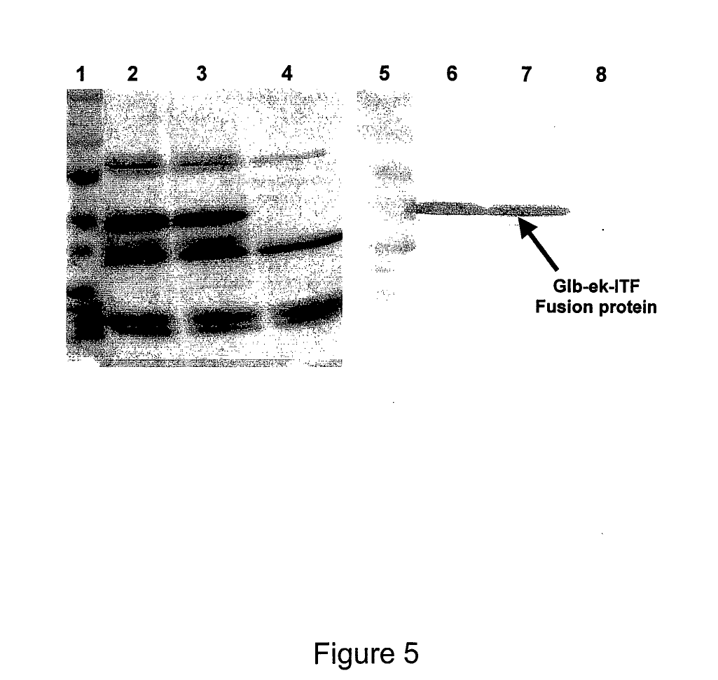

FIG. 5 shows the detection of the ITF moiety in the Glb-ek-ITF fusion protein by Western blot analysis. Two transgenic samples (pooled seed samples) and a TP309 non-transgenic sample were run onto two identical gels. One gel was Coomassie stained to visualize all proteins and the other gel was probed with a specific anti-ITF antibody. The fusion protein bands visualized in the Coomassie stained gel were detected by the antibody in the Western blot thus confirming the expression of mature ITF as a fusion protein in recombinant rice grains.

The present invention allows the expression of a fusion construct comprising a small heterologous peptide or polypeptide and a monocot seed storage protein, optionally including a methionine or tryptophan residue engineered in frame between the small heterologous peptide or polypeptide and the monocot seed storage protein. Expression of such a fusion construct has reached a level >100 μg/grain in transgenic rice seeds. Besides AOD, the successful method of the invention allows for expression of a variety of peptides of nutritional, pharmacological and medical importance. These include, but are not limited to: peptides for treating obesity such as PYY, peptide antibiotics such as iseganan and β-defensin, mature peptide growth factors such as EGF, IGF, FGF and ITF, anti-HIV peptides such as Fuzeon and derivatives, peptide hormones and peptide hormone fragments such as parathyroid hormone (PTH), adrenocorticotropin (ACTH) and gastrin-releasing peptide (GRP) and peptides for treating hypertension such as vasoactive intestinal peptide (VIP) and vascular endothelial growth inhibitor (VEGI). This specific fusion strategy may also be utilized for high-level expression of antigenic polypeptide epitopes specific for a variety of bacterial and viral diseases that may be used for oral immunization against these diseases.

### Rice Globulin as a Seed Storage Protein Fusion Partner

Two dimensional gel electrophoresis of rice seed storage protein extracts indicates that the 19 kDa globulin protein is largely, if not entirely, a single component and does not appear to exist as a family of proteins. Although the content in rice endosperm of the 19 kDa globulin protein is roughly 10% of the glutelin protein content, the 19 kDa globulin may be the most abundant product of a single gene in rice endosperm and in this respect, is an excellent choice to manipulate as a fusion carrier for heterologous peptide expression in rice endosperm. The globulin gene has previously been isolated and characterized and the DNA sequence determined. Other monocot seed storage proteins that may be used as potential fusion partners for high-level expression of heterologous peptides include rice glutelins, oryzins, and prolamines, barley hordeins, wheat gliadins and glutenins, maize zeins and glutelins, oat glutelins, sorghum kafirins, millet pennisetins, and rye secalins.

### EXAMPLE 4

### Human AOD9604 Sequence and Plasmid Construction

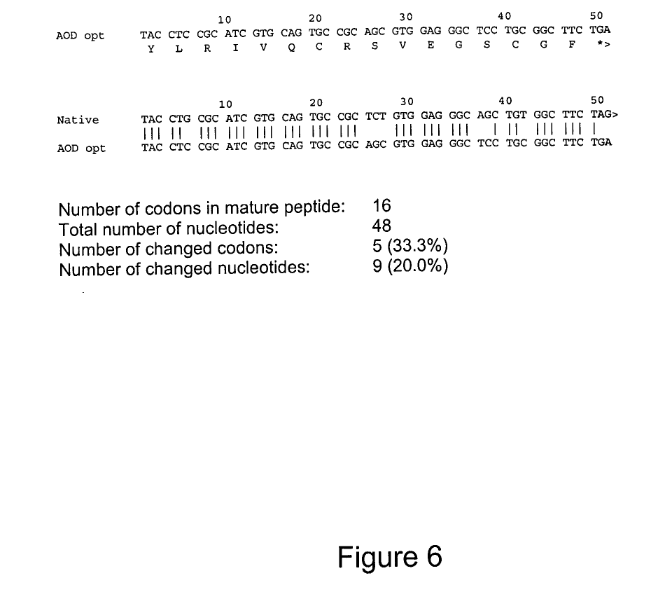

Human AOD9604 DNA sequence was based on the C-terminal fragment of human growth hormone (Natera et al., Biochem. Mol. Biol. Int. 33, 1011-1021, 1994). The sequence encodes an open reading frame for the 16 amino acid AOD peptide and was provided by Metabolics Ltd (Melbourne, AUS). For expression of AOD in rice grain, DNA sequence encoding the 16 amino acid AOD peptide was codon-optimized (FIG. 6) based on a codon-table specific for the expression of endogenous rice genes.

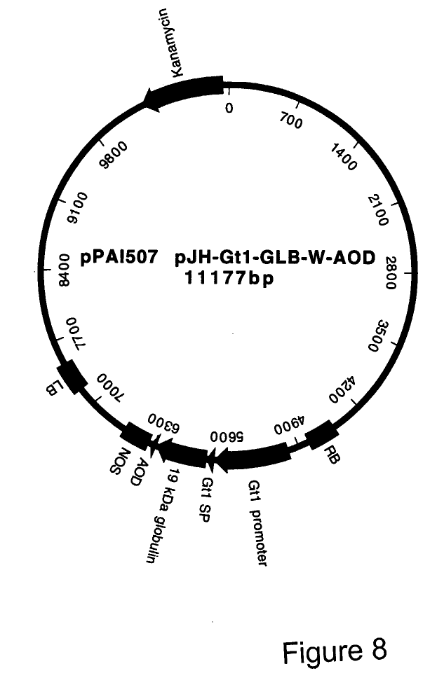

Three recombinant DNAs were prepared to express AOD in rice grain. First, an entire synthetic gene was synthesized containing the mature portion of the globulin storage protein (GLB), a tryptophan residue and the AOD9604 peptide (using rice-preferred codons). This synthetic gene encodes the GLB-W-AOD fusion protein. In addition, the sole tryptophan residue in the native mature globulin protein was converted to a proline residue (amino acid position 127) in this GLB-W-AOD fusion protein (FIG. 8) to eventually facilitate chemical release of the AOD peptide from the globulin fusion carrier by N-chlorosuccinimide at the newly introduced tryptophan residue at C-terminal end of the mature globulin protein (FIG. 8).

The GLB-W-AOD gene fragment was excised with the restriction enzymes Pml and Xho and this blunt-end/Xho DNA segment containing the GLB-W-AOD gene was isolated from a 1% agarose gel and purified using QIAGEN gel extraction protocol. The Gt1 promoter/signal peptide expression cassette containing plasmid, pAPI405 was digested with NaeI/XhoI and the vector DNA was also isolated on 1% agarose gel and purified using QIAGEN gel extraction protocol. The two DNA fragments were ligated with T4 DNA ligase and used to transform competent E. coli cells. The resulting plasmid (pAPI506) contained the rice Gt1 promoter, Gt1 signal peptide, the GLB-W-AOD fusion protein coding region and nos terminator 3′ region. The entire expression cassette (Gt1 promoter/Gt1sp:GLB-W-AOD fusion protein/nos terminator region) was excised from plasmid pAPI506 via the enzymes HindIII and EcoRI and cloned into the binary vector plasmid pJH2600 (Horvath et al, Proc. Natl. Acad. Sci. 97, 1914-1919, 2000) at these same restriction sites to form the binary plasmid pAPI507, containing the entire expression cassette (FIG. 8).

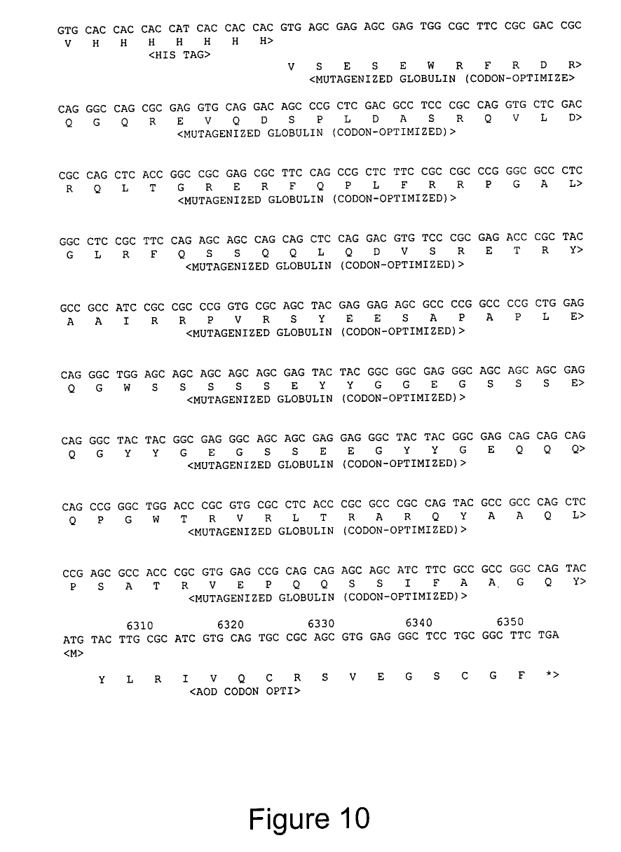

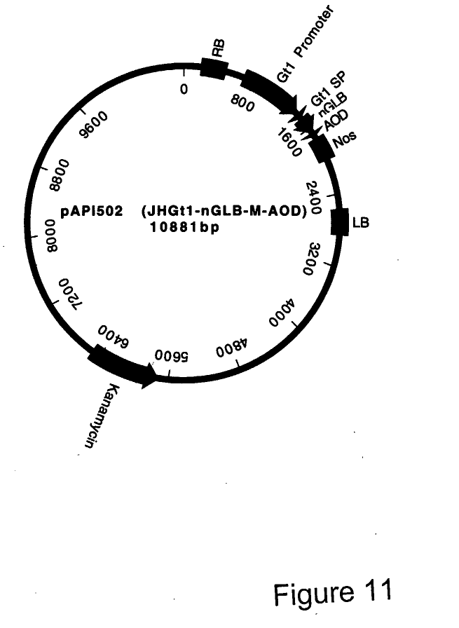

The second fusion of N-terminal of globulin gene was synthesized with rice prefer codons. A tryptophan was engineered between a fusion and AOD for releasing AOD from the fusion by chemical cleavage (FIG. 10). The synthesized gene fragment digested by SchI/XhoI and then directly cloned into pAPI405 digested by NaeI/XhoI to generate the intermediate plasmid, pAPI500. A fragment containing an entire expression cassette and fusion/AOD from pAPI500 was excised by HindIII and EcoRI and cloned into the binary vector plasmid pJH2600 at these same restriction sites to form the binary plasmid pAPI502, containing the entire expression cassette (FIG. 11).

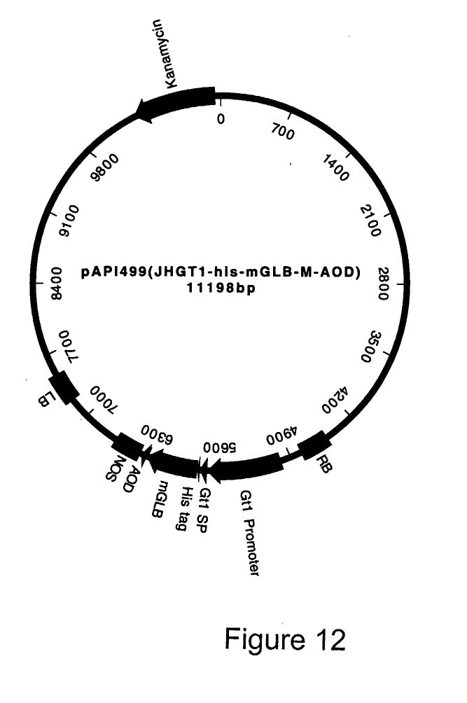

The third fusion carrier is mutated globulin gene. All methionines were mutated to serines to eliminate a cleavage site by cyanogen bromide and all cysteins were mutated to glycines to eliminate the disulfide bonds and a His6 tag was linked into the N-terminal of the fusion partner for future purification purpose. An additional methionine was put between the fusion and AOD to create cleavage site by cyanogen bromide. The fragment was synthesized by Blue Heron Technologies (FIG. 11). The synthesized fragment was excised with restriction enzymes PmI and XhoI, and cloned into Gt1 promoter/signal expression cassette (pAPI405) to generate the intermediate plasmid, pAPI494. A fragment containing an entire expression cassette and fusion/AOD from pAPI494 was excised by HindIII and EcoRI and cloned into the binary vector plasmid pJH2600 at these same restriction sites to form the binary plasmid pAPI499, containing the entire expression cassette (FIG. 12).

### EXAMPLE 5

### Rice Transformation and Plant Regeneration

A selectable marker plasmid pAPI412, consisting of phosphinothricin acetyltranferase (Bar) gene, driven by the Gns9 promoter and followed by the nos terminator, which is flanked by right and left borders of T-DNA in a binary vector, JH2600, provided the selectable marker DNA segment for all plant transformations. Plasmids pAPI412 and pAPI507, pAPI499 and pAPI502 were independently transformed into Agrobacterium strain LBA4404 and the Agrobacterium strains containing the individual plasmids were mixed in a 1:1 ratio after overnight growth on selective media. Agrobacterium-mediated transformation of rice was essentially carried out according to the procedure described in U.S. Pat. No. 5,591,616. Rice calli obtained from mature rice embryos were prepared for transformation as described in Huang et al. Rice calli derived from rice variety TP309 was inoculated with Agrobacterium LBA4404 containing plasmids pAPI412 and AOD plasmids. After 3 days co-cultivation, the calli were transferred to a selective medium containing 5 mg/l Bialaphos for 8-9 weeks. The surviving calli were regenerated into the entire plants on regeneration and then on the rooting medium. Transgenic plants (Table 1 below) were raised to maturity in the greenhouse and R1 seed collected for expression analysis.

TABLE 1
Total transgenic plants obtained from three constructs

| | Gt1- | Gt1- | | |
|---|---|---|---|---|
| | mGLB-M- | nGLB-M- | Gt1-GLB- | |
| | AOD | AOD | W-AOD | |
| Constructs | (pAPI499) | (pAPI502) | (pAPI507) | Total |
| No. of transgenic plants | 336 | 320 | 441 | 1097 |
| No. of AOD PCR positive | 172 | 164 | 160 | 496 |
| transgenic plants | | | | |
| Co-transformation | 51.2 | 51.3 | 36.3 | 45.2 |
| frequency (%) | | | | |

### EXAMPLE 6

### Analysis of AOD-Containing Fusion Protein Expression in Mature Rice Grains

For protein extraction, individual dehusked R1 rice grains from transgenic plants containing construct of AOD-fusion protein were placed in wells of a grinding plate. To each well was added 0.2 ml of extraction buffer, Tris-buffered saline (TBS) plus 0.35M NaCl. The grains were ground using a Genome Grinder at 300 strokes/min for 12 min. The resulting seed extracts were centrifuged at 4000 rpm for 20 min and the seed supernatants were transferred to a new plate.

Alternatively, 10 dehusked rice grains were pooled and ground with a mortar and pestle in 2 ml extraction buffer, TBS plus 0.35M NaCl and then mixed for 1.5 hr at 37° C. The mixed slurry was centrifuged at 12000 rpm for 12 min and the supernatant transferred to a 2 ml Eppendrof tube and stored in −20° C. for future analysis.

For expression level analysis, a total of 32 μl (about 50-60 μg total protein) of individual seed supernatants were resolved on 4-20% pre-cast polyacrylamide gels (Novex, Carlsbad, Calif.) and the gel was stained with staining solution, 0.1% Coomassie Brilliant Blue R-250 and then destained to visualize protein bands. For Western blot analysis, the gel was electro-blotted to a 0.45 um nitrocellulose membrane, blocked with 5% non-fat dry milk in PBS for 3 hr and then rinsed in phosphate-buffered saline (PBS). For incubation with primary antibody, a mouse monoclonal antibody against AOD and globulin were used at 1:1000 dilution in a primary antibody solution, 5% BSA in PBS containing 0.05% Tween20 and the blot was incubated in the solution for overnight.

The resulting blot was washed with PBS three times for 10 min each. The secondary antibody (goat anti-rabbit IgG-alkaline phosphatase conjugate (Bio-Rad, CA) was 1:4000 diluted in blocking buffer. The membrane was then incubated in the secondary antibody solution for 2 h and then washed three times in PBS. Color development was initiated by adding the substrate BCIP-NBT (Sigma, St. Louis, Mo.), and the process was terminated by rinsing the blot with H₂O once the desirable intensity of the bands had been achieved.

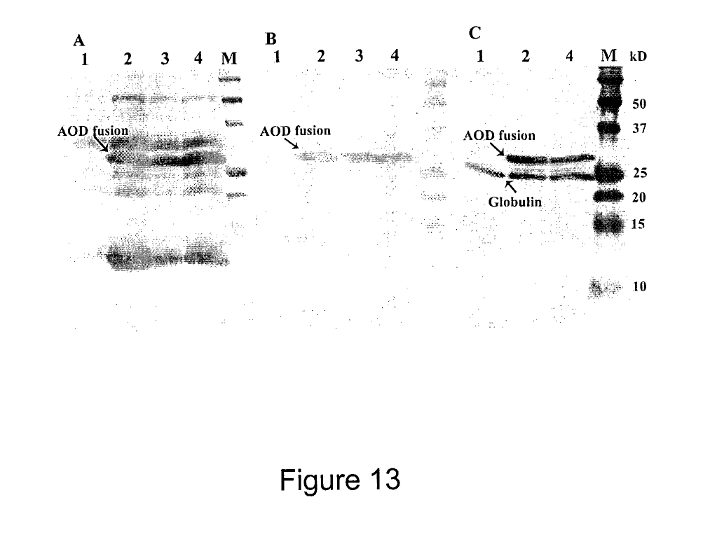

FIG. 13 (Gel B) shows the expression of GLB-W-AOD fusion protein resolved by Coomassie stained PAGE. Lane TP309 is the non-transgenic control in all gels. Extracts from two individual seed samples from transgenic events 507-13 and 507-17 are shown. GLB-W-AOD fusion protein is indicated by the arrow in all gels (Fusion). This band is not present in control TP309 lanes. FIG. 13 (Gel C) also shows the detection of the AOD moiety as a GLB-W-AOD fusion protein by Western analysis. The two transgenic pooled seed samples (507-13 and 507-17) along with a TP309 non-transgenic sample were run, Western blotted and he fusion protein visualized by anti-AOD antiserum. The fusion protein bands were also visualized by Western blotting using a globulin-specific antibody (Gel A) in the Western blot thus confirming the expression of the AOD peptide as a GLB fusion protein in recombinant rice grains. Initial expression estimates for the fusion protein in rice grains are 100-150 μg/seed. This translates into 0.5-0.75% of grain weight. As the fusion protein is about 1/10 the size of the mature globulin carrier, expression of AOD9604 peptide is roughly 0.05-0.075% of total grain weight.

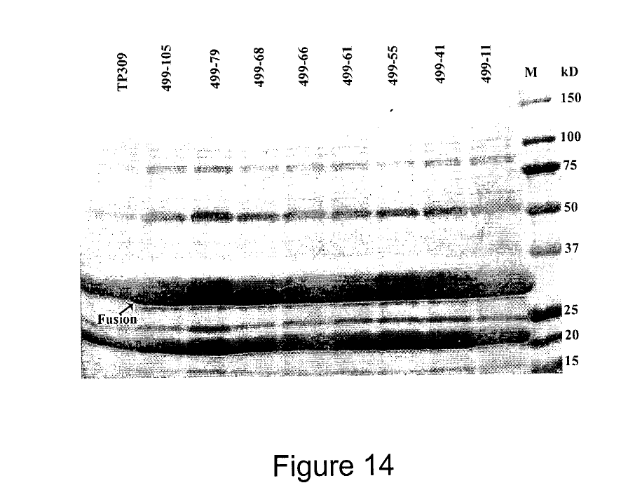

The inventors screened the transgenic plants produced from the construct pAPI449 using the same method. SDS-PAGE Coomassie-stained gel was conducted and for this construct, a total of 70 plants were detected to express His6-mGLB-AOD fusion. The top seven plant lines that had the highest expression of AOD9604 fusion protein from this construct are shown in FIG. 14. The expression level of the best line of plants for this construct, 499-105, was estimated at 5.6 mg/g flour or 0.56% of grain weight. Because the AOD9604 fusion protein in this construct contains a His tag, the molecular mass is a little higher than that of the AOD9604 fusion in the pAPI507 construct. The fusion protein has overlapped with a native protein that has the same molecular mass (FIG. 14). Thus there is a possibility that the expression level could be over-estimated for this line, although the background from the negative control parent line (TP309) was subtracted using Kodak gel documentation software.

For the construct pAPI502, 118 out of 164 transgenic plants were screened by SDS-PAGE gel. The nGLB-AOD fusion was detected by Western blot analysis, though it was difficult to see the nGLB-AOD fusion in the Coomassie staining gel. When analyzed using Western blot analysis, 48 transgenic plants had a positive signal (FIG. 14). The expression level of the nGLB-AOD9604 fusion in the best plant line from this construct is estimated at 15 μg/g flour. This demonstrated that this fusion approach does not produce high expression levels for AOD9604 when compared to the other two fusion partners.

### EXAMPLE 7

### Human Insulin-Like Growth Factor-1 (IGF-1) Sequence and Plasmid Construction

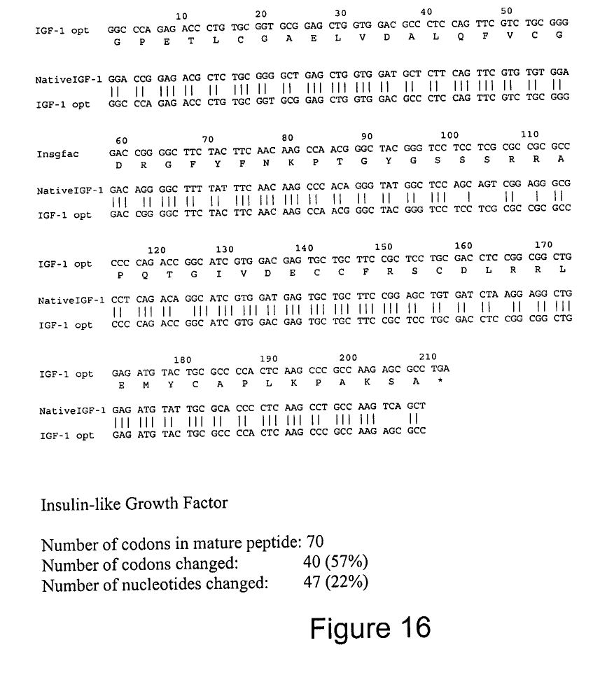

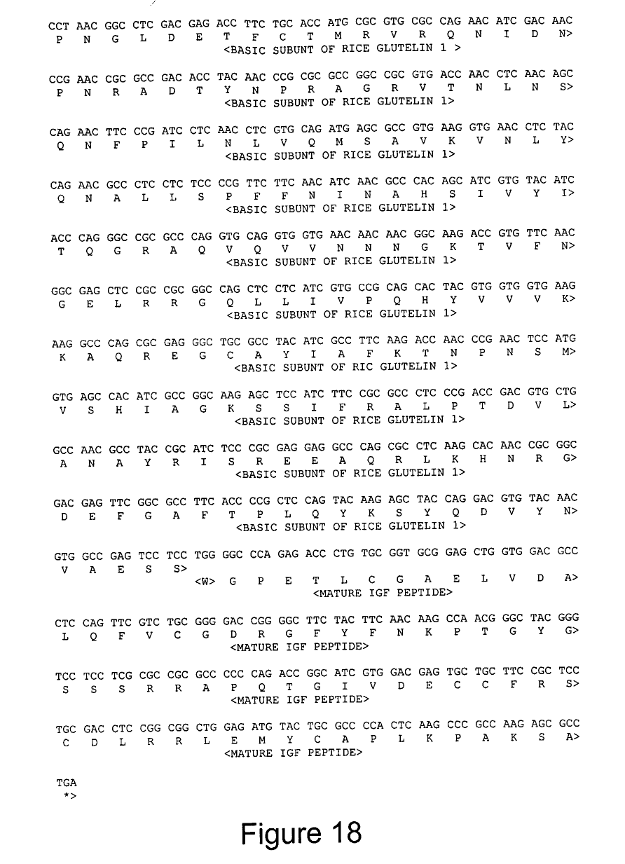

Human IGF-1 DNA sequence was based on GenBank protein sequence of GenBank accession number M11568. The sequence encodes an open reading frame for the 70 amino acid peptide. For expression of IGF-1 in rice grain, DNA sequence encoding the 70 amino acid IGF-1 peptide was codon-optimized (FIG. 16) based on a codon-table specific for the expression of endogenous rice genes. Two recombinant DNAs were prepared to express IGF-1 in rice grain. First, an entire synthetic gene was synthesized containing the mature portion of the globulin storage protein (GLB), a tryptophan residue and the IGF-1 peptide (using rice-preferred codons). This synthetic gene encoded the GLB-W-IGF-1 fusion protein. In addition, the sole tryptophan residue in the native mature globulin protein was converted to a proline residue (amino acid position 127) in this GLB-W-IGF-1 fusion protein (FIG. 18) to eventually facilitate chemical release of the IGF-1 peptide from the globulin fusion carrier by N-chlorosuccinimide at the newly introduced a tryptophan residue at C-terminal end of the mature globulin protein (FIG. 18).

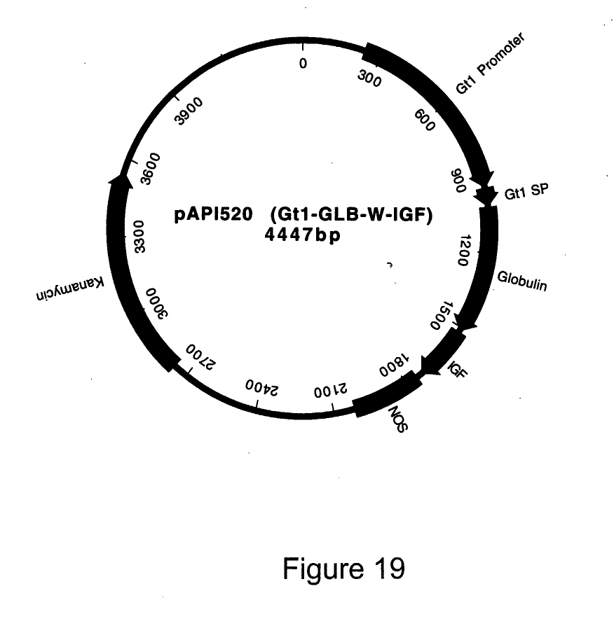

The GLB-W-IGF-1 gene fragment was excised with the restriction enzymes PmI and Xho and this blunt-end/Xho DNA segment containing the GLB-W-IGF-1 gene was isolated from a 1% agarose gel and purified using QIAGEN gel extraction protocol. The Gt1 promoter/signal peptide expression cassette containing plasmid, pAPI405 was digested with NaeI/XhoI and the vector DNA was also isolated on 1% agarose gel and purified using QIAGEN gel extraction protocol. The two DNA fragments were ligated with T4 DNA ligase and used to transform competent E. coli cells. The resulting plasmid contained the rice Gt1 promoter, Gt1 signal peptide, the GLB-W-IGF-1 fusion protein coding region and nos terminator 3′ region (FIG. 19).

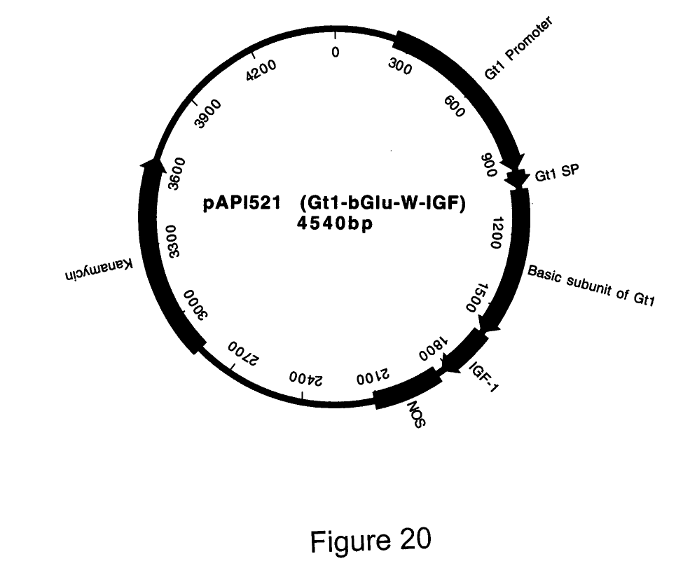

The second fusion partner is a basic subunit of glutelin. This fragment with a tryptophan residue between the fusion partner and IGF was synthesized by Blue Heron Technologies with rice prefer codons (FIG. 18). The fragment was excised by PmI and XhoI and cloned into pAPI405, resulting in plasmid pAPI521 (FIG. 20).

### EXAMPLE 8

### Rice Transformation and Plant Regeneration

Approximately 200 TP309 seeds were dehusked, sterilized in 50% v/v commercial bleach for 25 min and washed with sterile water three times for 5 min each. Sterilized seeds were placed on seven plates containing N6 media supplemented with 2 mg/l 2,4-D for 10 days to induce calli. The primary calli were dissected and placed on fresh N6 media for three weeks. The secondary calli were separated from the primary calli and placed on same N6 media to generate the tertiary calli. The tertiary calli were used for bombardment or sub-cultured 4-5 times every two weeks. The callus from each subculture can be used for bombardment.

Calli of 1 to 4 mm in diameter were selected and placed in a 4 cm circle on N6 media with 0.3 M mannitol and 0.3 M sorbitol for 5-24 h before bombardment. Biolistic bombardment was carried out with the Biolistic PDC-1000/He system (Bio-Rad). The procedure required 1.5 mg of gold particles (60 μg/μl) coated with 2.5 μg selectable marker DNA and co-transferred plasmid DNA (pAPI520 or pAPI521) at a ratio of 1 to 3. DNA-coated gold particles were bombarded into the rice callus with a helium pressure of 1100 psi. After bombardment, the calli were allowed to recover on the same plate for 48 hrs and then transferred to N6 media with 50 mg/L Hygromycin B.

The bombarded calli were incubated on the selection media in the dark at 26° C. for 45 days. At this time, transformants, which were white, opaque, compact and easily distinguished from the non-transformants which appear to be yellowish or brown, soft, and watery, were then transferred to the regeneration media consisting of N6 (without 2,4-D) 3 mg/l BAP, and 1 mg/l NAA without Hygromycin B and cultured under continuous lighting conditions for about two to three weeks.

When the regenerated plants were 1 to 3 cm high, the plantlets were transferred to the rooting media which was half the concentration of the MS media and contained 0.05 mg/l NAA. In two weeks, the plantlets in the rooting media developed roots and its shoots grew over 10 cm. The plants were then transferred to a 2.5 inch pot containing 50% commercial soil, Sunshine #1 (Sun Gro Horticulture Inc, WA) and 50% natural soil from rice fields. The pots were placed within a plastic container which was covered by another transparent plastic container to maintain higher humidity. The plants were cultured under continuous light for 1 week. The transparent plastic cover was then shifted slowly during one day period to gradually reduce the humidity. Afterwards, the plastic cover was removed completely, and water and fertilizers were added as necessary. When the plants grew to approximately 12 cm tall, they were transferred to a greenhouse where they grew to maturity.

It is to be understood that while the invention has been described above using specific embodiments, the description and examples are intended to illustrate the structural and functional principles of the present invention and are not intended to limit the scope of the invention. On the contrary, the present invention is intended to encompass all modifications, alterations, and substitutions within the spirit and scope of the appended claims.

What is claimed is:

1. A method of producing monocot seeds exhibiting expression of a heterologous peptide or polypeptide, comprising:
(a) transforming a monocot plant cell with a chimeric gene comprising:
(i) a promoter that is active in plant cells;
(ii) an optional first DNA sequence, operably linked to the promoter, encoding a signal sequence;
(iii) a second DNA sequence, operably linked to the promoter, encoding a monocot seed storage protein; and
(iv) a third DNA sequence, operably linked to the promoter, encoding a heterologous peptide or polypeptide,
wherein the optional first, second, and third DNA sequences are linked in translation frame and together encode a fusion protein comprising the optional signal sequence, the monocot seed storage protein, and the heterologous peptide or polypeptide;
(b) growing a monocot plant from the transformed monocot plant cell for a time sufficient to produce seeds containing the fusion protein; and
(c) harvesting the seeds from the monocot plant.

2. The method of claim 1, wherein the monocot plant is selected from corn, rice, barley, wheat, rye, corn, millet, triticale, or sorghum.

3. The method of claim 2, wherein the monocot plant is rice.

4. The method of claim 1, wherein the heterologous peptide or polypeptide is about 10 kDa or less.

5. The method of claim 1, wherein the heterologous peptide or polypeptide is between 5 and 100 amino acids in length.

6. The method of claim 1, wherein the chimeric gene further comprises a fourth DNA sequence, operably linked to the promoter, encoding a methionine or tryptophan residue and the fusion protein further comprises the methionine or tryptophan residue engineered in frame between the heterologous peptide or polypeptide and the monocot seed storage protein.

7. The method of claim 1, further comprising cleaving the fusion protein to separate the heterologous peptide or polypeptide from the monocot seed storage protein.

8. The method of claim 7, wherein the chimeric gene further comprises a fourth DNA sequence, operably linked to the promoter, encoding at least one selective purification tag and/or at least one specific protease cleavage site, and the fusion protein further comprises the at least one selective purification tag and/or at least one specific protease cleavage site fused in translation frame between the heterologous peptide or polypeptide and the monocot seed storage protein.

9. The method of claim 8, further comprising cleaving the fusion protein to separate the heterologous peptide or polypeptide from the monocot seed storage protein.

10. The method of claim 8, wherein the at least one specific protease cleavage site is enterokinase, Factor Xa, thrombin, V8 protease, Genenase™, α-lytic protease or tobacco etch virus protease.

11. The method of claim 10, wherein the at least one specific protease cleavage site is enterokinase.

12. The method of claim 7, wherein the fusion protein is cleaved by a chemical cleaving agent.

13. The method of claim 12, wherein the chemical cleaving agent is cyanogen bromide.

14. A transformed monocot plant cell, comprising:
a) a promoter that is active in plant cells;
b) an optional first DNA sequence, operably linked to the promoter, encoding a signal sequence;
c) a second DNA sequence, operably linked to the promoter, encoding a monocot seed storage protein; and
d) a third DNA sequence, operably linked to the promoter, encoding a heterologous peptide or polypeptide,
wherein the optional first, second, and third DNA sequences are linked in translation frame and together encode a fusion protein comprising the optional signal sequence, the storage protein, and the heterologous peptide or polypeptide.

15. The transformed monocot plant cell of claim 14, wherein the monocot plant is selected from corn, rice, barley, wheat, rye, corn, millet, triticale, or sorghum.

16. The transformed monocot plant cell of claim 15, wherein the monocot plant is rice.

17. The transformed monocot plant cell of claim 14, wherein the heterologous peptide or polypeptide is about 10 kDa or less.

18. The transformed monocot plant cell of claim 14, wherein the heterologous peptide or polypeptide is between 5 and 100 amino acids in length.

19. The transformed monocot plant cell of claim 14, wherein the chimeric gene further comprises a fourth DNA sequence, operably linked to the promoter, encoding a methionine or tryptophan residue and the fusion protein further comprises the methionine or tryptophan residue engineered in frame between the heterologous peptide or polypeptide and the monocot seed storage protein.

20. The transformed monocot plant cell of claim 14, wherein the chimeric gene further comprises a fourth DNA sequence, operably linked to the promoter, encoding at least one selective purification tag and/or at least one specific protease cleavage site, and the fusion protein further comprises the at least one selective purification tag and/or at least one specific protease cleavage site fused in translation frame between the heterologous peptide or polypeptide and the monocot seed storage protein.

21. The transformed monocot plant cell of claim 20, wherein the at least one specific protease cleavage site is enterokinase, Factor Xa, thrombin, V8 protease, Genenase™, α-lytic protease or tobacco etch virus protease.

22. The transformed monocot plant cell of claim 21, wherein the at least one specific protease cleavage site is enterokinase.

23. A chimeric gene, comprising:
a) a promoter that is active in plant cells;
b) an optional first DNA sequence, operably linked to the promoter, encoding a signal sequence;
c) a second DNA sequence, operably linked to the promoter, encoding a monocot seed storage protein; and
d) a third DNA sequence, operably linked to the promoter, encoding a heterologous peptide or polypeptide,
wherein the optional first, second, and third DNA sequences are linked in translation frame and together encode a fusion protein comprising the optional signal sequence, the storage protein, and the heterologous peptide or polypeptide.

24. The chimeric gene of claim 23, wherein the monocot plant is corn, rice, barley, wheat, rye, corn, millet, triticale, or sorghum.

25. The chimeric gene of claim 24, wherein the monocot plant is rice.

26. The chimeric gene of claim 23, wherein the heterologous peptide or polypeptide is about 10 kDa or less.

27. The chimeric gene of claim 23, wherein the heterologous peptide or polypeptide is between 5 and 100 amino acids in length.

28. The chimeric gene of claim 23, further comprising a fourth DNA sequence, operably linked to the promoter, encoding a methionine or tryptophan residue and the fusion protein further comprises the methionine or tryptophan residue engineered in frame between the heterologous peptide or polypeptide and the monocot seed storage protein.

29. The chimeric gene of claim 23, wherein the chimeric gene further comprises a fourth DNA sequence, operably linked to the promoter, encoding at least one selective purification tag and/or at least one specific protease cleavage site, and the fusion protein further comprises the at least one selective purification tag and/or at least one specific protease cleavage site fused in translation frame between the heterologous peptide or polypeptide and the monocot seed storage protein.

30. The chimeric gene of claim 29, wherein the at least one specific protease cleavage site is enterokinase, Factor Xa, thrombin, V8 protease, Genenase™, 60 -lytic protease or tobacco etch virus protease.

31. The chimeric gene of claim 30, wherein the at least one specific protease cleavage site is enterokinase.

32. A method of expressing a heterologous peptide or polypeptide in a monocot plant seed, the method comprising:
a) fusing a heterologous peptide or polypeptide with a monocot seed storage protein in a monocot mature seed expression system, and
b) expressing the heterologous peptide or polypeptide in the mature monocot seed.

33. The method of claim 32, wherein the expression of the heterologous peptide or polypeptide in the monocot plant seed is at least a 20-fold greater than the expression of the heterologous peptide or polypeptide in the absence of the seed-storage protein.

34. The method of claim 32, wherein the heterologous peptide or polypeptide is expressed at a level of at least 15-20 μg/monocot plant seed.

35. The method of claim 32, wherein the heterologous peptide or polypeptide is at least 3.0% of total soluble protein of the seed.

36. The method of claim 35, wherein the heterologous peptide or polypeptide is at least 5.0% of total soluble protein of the seed.


SEQUENCE LISTING (21 sequences)

SEQ ID NO: 1 - DNA, 183 bp, Artificial Sequence
  CDS (1)..(180)
  Description of Artificial Sequence Synthetic DNA construct
gaggagtacgtcgggctctccgctaaccaatgcgcggtcccggccaaggaccgggtggactgcggctacccccacgtgacgccgaaggagtgcaacaaccggggctgctgcttcgactcccgcatcccaggcgtgccgtggtgcttcaagcccctcacccgcaagacggagtgcacgttctga

SEQ ID NO: 2 - protein, 60 aa, Artificial Sequence
  Description of Artificial Sequence Synthetic amino acid construct
EEYVGLSANQCAVPAKDRVDCGYPHVTPKECNNRGCCFDSRIPGVPWCFKPLTRKTECTF

SEQ ID NO: 3 - DNA, 183 bp, Homo sapiens
gaggagtacgtgggcctgtctgcaaaccagtgtgccgtgccggccaaggacagggtggactgcggctacccccatgtcacccccaaggagtgcaacaaccggggctgctgctttgactccaggatccctggagtgccttggtgtttcaagcccctgactaggaagacagaatgcaccttctga

SEQ ID NO: 4 - DNA, 762 bp, Artificial Sequence
  CDS (1)..(759)
  Description of Artificial Sequence Synthetic DNA construct
atggcatccataaatcgccccatagttttcttcacagtttgcttgttcctcttgtgcgatggctccctagcccacgtgagcgagtcggagatgaggttcagggacaggcagtgccagcgggaggtgcaggacagcccgctggacgcgtgccggcaggtgctcgaccggcagctcaccggccgggagaggttccagccgatgttccgccgcccgggcgcgctcggcctgcggatgcagtgctgccagcagctgcaggacgtgagccgcgagtgccgctgcgccgccatccgccggatggtgaggagctacgaggagagcatgccgatgcccctggagcaaggctggtcgtcgtcgtcgtcggagtactacggcggcgaggggtcgtcgtcggagcaggggtactacggcgaggggtcgtcggaggagggctactacggcgagcagcagcagcagccggggatgacccgcgtgaggctgaccagggcgaggcagtacgcggcgcagctgccgtcgatgtgccgggttgagccccagcagtgcagcatcttcgccgccggccagtacgacgacgacgacaaggaggagtacgtgggcctcagcgccaaccagtgcgccgtgccggccaaggaccgcgtggactgcggctacccgcacgtgaccccgaaggagtgcaacaaccgcggctgctgcttcgacagccgcatcccgggcgtgccgtggtgcttcaagccgctcacccgcaagaccgagtgcaccttctga

SEQ ID NO: 5 - protein, 253 aa, Artificial Sequence
  Description of Artificial Sequence Synthetic amino acid construct
MASINRPIVFFTVCLFLLCDGSLAHVSESEMRFRDRQCQREVQDSPLDACRQVLDRQLTGRERFQPMFRRPGALGLRMQCCQQLQDVSRECRCAAIRRMVRSYEESMPMPLEQGWSSSSSEYYGGEGSSSEQGYYGEGSSEEGYYGEQQQQPGMTRVRLTRARQYAAQLPSMCRVEPQQCSIFAAGQYDDDDKEEYVGLSANQCAVPAKDRVDCGYPHVTPKECNNRGCCFDSRIPGVPWCFKPLTRKTECTF

SEQ ID NO: 6 - DNA, 51 bp, Artificial Sequence
  CDS (1)..(48)
  Description of Artificial Sequence Synthetic DNA construct
tacctccgcatcgtgcagtgccgcagcgtggagggctcctgcggcttctga

SEQ ID NO: 7 - protein, 16 aa, Artificial Sequence
  Description of Artificial Sequence Synthetic amino acid construct
YLRIVQCRSVEGSCGF

SEQ ID NO: 8 - DNA, 51 bp, Homo sapiens
tacctgcgcatcgtgcagtgccgctctgtggagggcagctgtggcttctag

SEQ ID NO: 9 - DNA, 615 bp, Artificial Sequence
  CDS (1)..(612)
  Description of Artificial Sequence Synthetic DNA construct
atggcatccataaatcgccccatagttttcttcacagtttgcttgttcctcttgtgcgatggctccctagccgtgagcgagtccgagatgcgcttccgcgaccgccagtgccagcgcgaggtgcaggacagcccgctcgacgcctgccgccaggtgctcgaccgccagctcaccggccgcgagcgcttccagccgatgttccgccgcccgggcgcgctcggcctccgcatgcagtgctgccagcagctccaggacgtgagccgcgagtgccgctgcgccgccatccgccgcatggtgcgcagctacgaggagagcatgccgatgccgctggagcagggcccgtcctcctccagcagcgagtactacggcggcgagggctccagctccgagcagggctactacggcgagggctcctccgaggagggctactacggcgagcagcagcagcagccgggcatgacccgcgtgcgcctcacccgcgcccgccagtacgccgcccagctcccgtccatgtgccgggtggagccgcagcagtgcagcatcttcgccgccggccagtactggtacctccgcatcgtgcagtgccgcagcgtggagggctcctgcggcttctga

SEQ ID NO: 10 - protein, 204 aa, Artificial Sequence
  Description of Artificial Sequence Synthetic amino acid construct
MASINRPIVFFTVCLFLLCDGSLAVSESEMRFRDRQCQREVQDSPLDACRQVLDRQLTGRERFQPMFRRPGALGLRMQCCQQLQDVSRECRCAAIRRMVRSYEESMPMPLEQGPSSSSSEYYGGEGSSSEQGYYGEGSSEEGYYGEQQQQPGMTRVRLTRARQYAAQLPSMCRVEPQQCSIFAAGQYWYLRIVQCRSVEGSCGF

SEQ ID NO: 11 - DNA, 249 bp, Artificial Sequence
  CDS (1)..(246)
  Description of Artificial Sequence Synthetic DNA construct
cacgtgagcgagagcgagagcaggttcagggacaggcagtgccagcgggaggtgcaggacagcccgctggacgcgtgccggcaggtgctcgaccggcagctcaccggccgggagaggttccagccgtccttccgccgcccgggcgcgctcggcctgcggagccagtgctgccagcagctgcaggacgtgagccgcatgtacttgcgcatcgtgcagtgccgcagcgtggagggctcctgcggcttctga

SEQ ID NO: 12 - protein, 82 aa, Artificial Sequence
  Description of Artificial Sequence Synthetic amino acid construct
HVSESESRFRDRQCQREVQDSPLDACRQVLDRQLTGRERFQPSFRRPGALGLRSQCCQQLQDVSRMYLRIVQCRSVEGSCGF

SEQ ID NO: 13 - DNA, 567 bp, Artificial Sequence
  CDS (1)..(564)
  Description of Artificial Sequence Synthetic DNA construct
gtgcaccaccaccatcaccaccacgtgagcgagagcgagtggcgcttccgcgaccgccagggccagcgcgaggtgcaggacagcccgctcgacgcctcccgccaggtgctcgaccgccagctcaccggccgcgagcgcttccagccgctcttccgccgcccgggcgccctcggcctccgcttccagagcagccagcagctccaggacgtgtcccgcgagacccgctacgccgccatccgccgcccggtgcgcagctacgaggagagcgccccggccccgctggagcagggctggagcagcagcagcagcgagtactacggcggcgagggcagcagcagcgagcagggctactacggcgagggcagcagcgaggagggctactacggcgagcagcagcagcagccgggctggacccgcgtgcgcctcacccgcgcccgccagtacgccgcccagctcccgagcgccacccgcgtggagccgcagcagagcagcatcttcgccgccggccagtacatgtacttgcgcatcgtgcagtgccgcagcgtggagggctcctgcggcttctga

SEQ ID NO: 14 - protein, 188 aa, Artificial Sequence
  Description of Artificial Sequence Synthetic amino acid construct
VHHHHHHHVSESEWRFRDRQGQREVQDSPLDASRQVLDRQLTGRERFQPLFRRPGALGLRFQSSQQLQDVSRETRYAAIRRPVRSYEESAPAPLEQGWSSSSSEYYGGEGSSSEQGYYGEGSSEEGYYGEQQQQPGWTRVRLTRARQYAAQLPSATRVEPQQSSIFAAGQYMYLRIVQCRSVEGSCGF

SEQ ID NO: 15 - DNA, 213 bp, Artificial Sequence
  CDS (1)..(210)
  Description of Artificial Sequence Synthetic DNA construct
ggcccagagaccctgtgcggtgcggagctggtggacgccctccagttcgtctgcggggaccggggcttctacttcaacaagccaacgggctacgggtcctcctcgcgccgcgccccccagaccggcatcgtggacgagtgctgcttccgctcctgcgacctccggcggctggagatgtactgcgccccactcaagcccgccaagagcgcctga

SEQ ID NO: 16 - protein, 70 aa, Artificial Sequence
  Description of Artificial Sequence Synthetic amino acid construct
GPETLCGAELVDALQFVCGDRGFYFNKPTGYGSSSRRAPQTGIVDECCFRSCDLRRLEMYCAPLKPAKSA

SEQ ID NO: 17 - DNA, 210 bp, Homo sapiens
ggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagct

SEQ ID NO: 18 - DNA, 708 bp, Artificial Sequence
  CDS (1)..(705)
  Description of Artificial Sequence Synthetic DNA construct
cacgtgagcgagtcggagatgaggttcagggacaggcagtgccagcgggaggtggaggacagcccgctggacgcgtgccggcaggtgctcgaccggcagctcaccggccgggagaggttccagccgatgttccgccgcccgggcgcgctcggcctgcggatgcagtgctgccagcagctgcaggacgtgagccgcgagtgccgctgcgccgccatccgccggatggtgaggagctacgaggagagcatgccgatgcccctggagcaaggctggtcgtcgtcgtcgtcggagtactacggcggcgaggggtcgtcgtcggagcaggggtactacggcgaggggtcgtcggaggagggctactacggcgagcagcagcagcagccggggatgacccgcgtgaggctgaccagggcgaggcagtacgcggcgcagctgccgtcgatgtgccgggttgagccccagcagtgcagcatcttcgccgccggccagtactggggcccagagaccctgtgcggtgcggagctggtggacgccctccagttcgtctgcggggaccggggcttctacttcaacaagccaacgggctacgggtcctcctcgcgccgcgccccccagaccggcatcgtggacgagtgctgcttccgctcctgcgacctccggcggctggagatgtactgcgccccactcaagcccgccaagagcgcctga

SEQ ID NO: 19 - protein, 235 aa, Artificial Sequence
  Description of Artificial Sequence Synthetic amino acid construct
HVSESEMRFRDRQCQREVEDSPLDACRQVLDRQLTGRERFQPMFRRPGALGLRMQCCQQLQDVSRECRCAAIRRMVRSYEESMPMPLEQGWSSSSSEYYGGEGSSSEQGYYGEGSSEEGYYGEQQQQPGMTRVRLTRARQYAAQLPSMCRVEPQQCSIFAAGQYWGPETLCGAELVDALQFVCGDRGFYFNKPTGYGSSSRRAPQTGIVDECCFRSCDLRRLEMYCAPLKPAKSA

SEQ ID NO: 20 - DNA, 801 bp, Artificial Sequence
  Description of Artificial Sequence Synthetic DNA construct
  CDS (1)..(798)
cctaacggcctcgacgagaccttctgcaccatgcgcgtgcgccagaacatcgagaacccgaaccgcgccgacacctacaacccgcgcgccggccgcgtgaccaacctcaacagccagaacttcccgatcctcaacctcgtgcagatgagcgccgtgaaggtgaacctctaccagaacgccctcctctccccgttcttcaacatcaacgcccacagcatcgtgtacatcacccagggccgcgcccaggtgcaggtggtgaacaacaacggcaagaccgtgttcaacggcgagctccgccgcggccagctcctcatcgtgccgcagcactacgtggtggtgaagaaggcccagcgcgagggctgcgcctacatcgccttcaagaccaacccgaactccatggtgagccacatcgccggcaagagctccatcttccgcgccctcccgaccgacgtgctggccaacgcctaccgcatctcccgcgaggaggcccagcgcctcaagcacaaccgcggcgacgagttcggcgccttcaccccgctccagtacaagagctaccaggacgtgtacaacgtggccgagtcctcctggggcccagagaccctgtgcggtgcggagctggtggacgccctccagttcgtctgcggggaccggggcttctacttcaacaagccaacgggctacgggtcctcctcgcgccgcgccccccagaccggcatcgtggacgagtgctgcttccgctcctgcgacctccggcggctggagatgtactgcgccccactcaagcccgccaagagcgcctga

SEQ ID NO: 21 - protein, 266 aa, Artificial Sequence
  Description of Artificial Sequence Synthetic amino acid construct
PNGLDETFCTMRVRQNIENPNRADTYNPRAGRVTNLNSQNFPILNLVQMSAVKVNLYQNALLSPFFNINAHSIVYITQGRAQVQVVNNNGKTVFNGELRRGQLLIVPQHYVVVKKAQREGCAYIAFKTNPNSMVSHIAGKSSIFRALPTDVLANAYRISREEAQRLKHNRGDEFGAFTPLQYKSYQDVYNVAESSWGPETLCGAELVDALQFVCGDRGFYFNKPTGYGSSSRRAPQTGIVDECCFRSCDLRRLEMYCAPLKPAKSA